(19) United States
(12) Patent Application Publication
(10) Pub. No.: US 2026/0192003 A1
(43) Pub. Date: Jul. 9, 2026

(54) CHELATING COMPOUNDS AND METHODS OF USES THEREOF

(71) Applicant: THE REGENTS OF THE UNIVERSITY OF CALIFORNIA, Oakland, CA (US)

(72) Inventors: Joshua Woods, Berkeley, CA (US); Rebecca J. Abergel, Kensington, CA (US)

(73) Assignee: THE REGENTS OF THE UNIVERSITY OF CALIFORNIA, Oakland, CA (US)

(21) Appl. No.: 19/463,530

(22) Filed: Jan. 29, 2026

Related U.S. Application Data
(63) Continuation of application No. PCT/US2024/040867, filed on Aug. 2, 2024.
(60) Provisional application No. 63/517,568, filed on Aug. 3, 2023.

(51) Int. Cl. A61K 51/04; C07D 401/14

(52) U.S. Cl. CPC A61K 51/0497; A61K 51/0455; C07D 401/14

(57) ABSTRACT

The present invention provides for a metal chelator capable of binding a radioisotope or radionuclide, and bioconjugate thereof, and methods of making and using thereof.

### RELATED PATENT APPLICATIONS

This application claims priority to, and is a 35 U.S.C. § 111 (a) continuation of, PCT international application number PCT/US2024/040867 filed on Aug. 2, 2024, incorporated herein by reference in its entirety, which claims priority to, and the benefit of, U.S. provisional patent application Ser. No. 63/517,568 filed on Aug. 3, 2023, incorporated herein by reference in its entirety. Priority is claimed to each of the foregoing applications.

The above-referenced PCT international application was published as PCT International Publication No. WO 2025/030160 A1 on Feb. 6, 2025, which publication is incorporated herein by reference in its entirety.

### STATEMENT OF GOVERNMENTAL SUPPORT

The invention was made with government support under Contract No. DE-AC02-05CH11231 awarded by the U.S. Department of Energy. The government has certain rights in the invention.

### FIELD OF THE INVENTION

The present invention is in the field of metal chelators.

### BACKGROUND OF THE INVENTION

Thorium-227 (²²⁷Th) is an α-emitting radionuclide that has shown preclinical and clinical promise for use in targeted α-therapy (TAT), a type of molecular radiopharmaceutical treatment that harnesses high energy α particles to eradicate cancerous lesions. Despite these initial successes, there still exists a need for bifunctional chelators that can stably bind thorium in vivo.

Targeted alpha therapy (TAT), a molecular radiopharmaceutical approach in which an α-emitting radionuclide is administered in vivo for the treatment of metastatic lesions, has received attention as a promising cancer treatment stategy.¹ The clinical approval and use of radium-223 (²²³Ra) in the form of [²²³Ra]RaCl₂ has helped demonstrate the value of α-emitting radionuclides for therapy. Acting as a calcium mimic in the body, [²²³Ra]Ra²⁺ naturally localizes to the bone where it delivers damaging radiation in the form of high-energy α particles to bone metastases associated with castration-resistant prostate cancer. The low penetration range of a particles through biological tissue (≤100 μm) helps to limit adverse effects in the surrounding healthy tissue.^{2,3}

Given the clinical success of [²²³Ra]RaCl₂, there has been significant interest in applying α-emitting isotopes, in particular radiometals, to treat cancers in soft tissues. To selectively deliver the α-emitting radionuclide to the desired tissue or organ, it must be stably conjugated to a high-affinity tumor-targeting moiety via a bifunctional chelator, which is a molecule that contains both a metal chelating unit and a reactive functional group.^{4,5} An ideal bifunctional chelator must be able to covalently bind the desired targeting vector and form a complex with the desired radiometal with high thermodynamic and kinetic stabilities to prevent transchelation in vivo by endogenous biomolecules. In this context, attempts to develop bifunctional chelators for ²²³Ra with sufficient stability in vivo have been hampered by the poorly understood coordination chemistry of this element.^{6,7} These challenges have prompted exploration of alternative α-emitting radionuclides with more established coordination chemistry.

Among the radionuclides considered for TAT, thorium-227 (²²⁷Th) has shown significant promise for this application.⁸⁻¹¹ In its decay to stable lead-207 (²⁰⁷Pb), ²²⁷Th produces five high-energy α particles, which cause DNA double-strand breaks and subsequent cell death.^{12,13} The 18.7-day half-life (t_{1/2}) of ²²⁷Th is suitable for use with antibody-based targeting vectors that have a long circulation time in vivo. Another benefit of ²²⁷Th for targeted radiotherapy is that the γ-rays emitted by this radionuclide may be used to quantitatively asses the distribution of the administered radiotherapeutic agent and the progress of the treatment in vivo using single-photon emission computed tomography/computed tomography (SPECT/CT) imaging.^{14,15} Lastly, ²²⁷Th is the parent isotope of ²²³Ra, meaning that established strategies currently used for the production of ²²³Ra can be directly applied to generation of ²²⁷Th,¹⁶⁻¹⁸ suggesting that sufficient quantities of this radioisotope may be available to sustain preclinical and clinical studies.

Despite the extensive knowledge of the aqueous chemistry of thorium^{19,20} and the promising radiological properties of ²²⁷Th for targeted radiotherapy, only a handful of chelators have been investigated for medical application of this isotope. In aqueous medium, thorium exists almost exclusively in the tetravalent oxidation state. The Th⁴⁺ ion is the largest stable tetravalent metal ion within the periodic table with an ionic radius of 0.94 Å (coordination number=6) and forms complexes with coordination numbers typically ranging from 5-12, reaching as high as 15.^{21,22} Additionally, Th⁴⁺ is highly oxophilic and readily undergoes hydrolysis to form insoluble hydroxide and oxide species in water.^{23,24} In vivo, Th⁴⁺ is readily incorporated into bone and soft tissues such as the kidney, liver, and spleen.^{25,26} Given the oxophilicity of Th⁴⁺, oxygen-rich chelators are the most promising candidates for chelation of this ion. The macrocyclic chelator 1,4,7,10-tetraazacyclododecane-N,N′,N″,N″-1,4,7,10-tetraacetic acid (DOTA) forms stable complexes with Th⁴⁺ in aqueous solution,²⁷⁻³⁰ but subsequent studies have shown poor radiolabeling yields with long reaction times and elevated temperatures,^{31,32} conditions that are not compatible with most biological targeting vectors. Ligands containing phosphonate donors form complexes with [²²⁷Th]Th⁴⁺ that are stable in vivo and enhance its uptake in bone compared to the unchelated metal,^{33,34} but these chelators have not been explored in the context of TAT. A terephthalamide (TAM)-based macrocycle with high affinity for Th⁴⁺ (log β_{ThL}=53.7) has been reported,³⁵ but the ability of this ligand to coordinate ²²⁷Th under radiolabeling conditions has not yet been explored. Picolinic acid “pa” chelators have recently been explored as chelators for several medically relevant isotopes of thorium. These ligands show promising stability in vivo, but an antibody-conjugated version of the ligand octapa showed only modest radiolabeling yields (<70%) at 37° C.^{36,37}

To accommodate the high coordination numbers and large ionic radius of Th⁴⁺, a number of octadentate and decadentate ligands containing 1-hydroxy-2-pyridinone (1,2-HOPO), 3-hydroxy-N-methyl-2-pyridinone (Me-3,2-HOPO), or catecholamide (CAM) chelating units have been explored for stable chelation of Th⁴⁺ in aqueous solution and in vivo.³⁸⁻⁴⁶ The current gold standard for chelation of ²²⁷Th is an octadentate ligand bearing four Me-3,2-HOPO groups (FIG. 10).^{47,48} This ligand, denoted here as L1, has a high thermodynamic affinity for Th⁴⁺ (log βThL=41.72) and quantitatively binds [²²⁷Th]Th⁴⁺ at room temperature. A series of preclinical studies where this ligand was conjugated to different tumor-targeting constructs highlight the therapeutic potential of ²²⁷Th and this chelator is currently in phase I clinical trials for treatment of tumors expressing mesothelin.⁴⁹⁻⁵³ Although L1 has shown promising preclinical results, its broader implementation in TAT potentially suffers from its challenging, multistep chemical synthesis. It would be beneficial to develop chelators that can be synthesized in a small number of steps from readily available starting materials.

As mentioned above, the γ-rays emitted by ²²⁷Th could be harnessed through SPECT/CT imaging to obtain important pharmacokinetic data relating to the biodistribution of this isotope in vivo and disease progression during treatment. In practice, however, this strategy is challenging due to the low yield of γ photons emitted from this radioisotope, requiring careful considerations for proper analysis.^{14,54} An alternative strategy to monitor the biokinetics of ²²⁷Th conjugates for TAT would be to pair its administration with a positron-emitting isotope, which can be tracked in the body using positron emission tomography (PET) imaging. In this context, it would be ideal to use radiometals that display similar coordination chemistries and can be bound by the same bioconjugate system in order to minimize differences in their localization in vivo.^{55,56} Zirconium-89 (⁸⁹Zr) is a positron-emitting isotope that has been suggested for use as a diagnostic isotope for ²²⁷Th.^{53,57,58} Both metals display comparable coordination preferences in aqueous solution, favoring oxygen-rich chelators with high denticity (CN>6).⁵⁹⁻⁶¹ As such, it is desirable to design a chelator that can stably coordinate both [⁸⁹Zr]Zr⁴⁺ and [²²⁷Th]Th⁴⁺ in vivo.

### SUMMARY OF THE INVENTION

The present invention provides for a metal chelator having a chemical structure comprising:

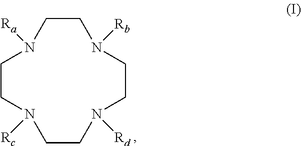

or a bioconjugate thereof; wherein Rₐ, R_{b}, R_{c}, and R_{d} each independently comprises:
(a) a HOPO group having the following chemical structure:

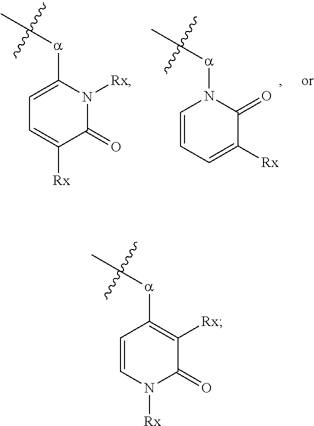

wherein α is a

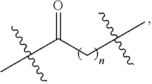

—CHOH—(CH₂)ₙ—, or —(CH₂)ₙ—; each Rₓ is independently a H, —OH or alkyl group; and n is an integer from 0 to 10;
(b) a HA group having the following chemical structure:

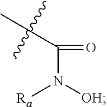

wherein Rα is a —H or alkyl group; or,

(c) a CAM group having the following chemical structure:

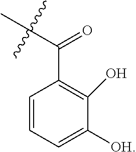 [SMILES: *C(=O)c1cccc(O)c1O]

In some embodiments, the HOPO group is

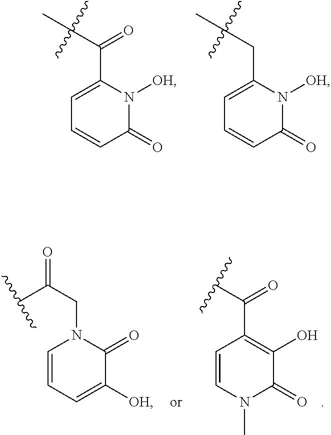 [SMILES: *C(=O)c1cccc(=O)n1O ; *Cc1cccc(=O)n1O ; *C(=O)Cn1cccc(O)c1=O ; *C(=O)c1ccn(C)c(=O)c1O]

In some embodiments, Rₐ, R_{b}, R_{c}, and R_{d} are identical. In some embodiments, each alkyl group is independently a branched or straight chain alkyl group. In some embodiments, each alkyl group is independently is —(CH₂)ₘ—CH₃, wherein m is an integer from 0 to 10. In some embodiments, α is

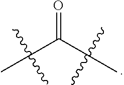 [SMILES: *C(*)=O]

In some embodiments, m and n are each independently an integer from 0 to 10, from 1 to 10, or from 1 to 5.

In some embodiments, the bioconjugated or bifunctional metal chelator comprising a -β-N═C═S covalently bound to the chemical structure (I), such as any of the carbon atoms of

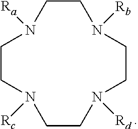

In some embodiments, the β comprises an aryl or alkyl group. In some embodiments, the alkyl group is a branched or straight chain alkyl group. In some embodiments, the alkyl group is independently is —(CH₂)ₖ—CH₃, wherein k is an integer from 0 to 10. In some embodiments, the aryl group is —(CH₂)ₒ—C₆H₄—(CH₂)ₚ—, wherein o and p are each independently an integer from 0 to 10. In some embodiments, the —C₆H₄— is

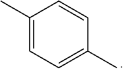

In some embodiments, k, o, and p are each independently an integer from 0 to 10, from 1 to 10, or from 1 to 5. In some embodiments, the -β-N═C═S is

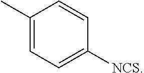

The NCS group is capable of reacting with a biological targeting vector (BTV) in order to covalently bind the metal chelator to the biological targeting vector. The NCS group reacts with an amine functional group to form a thiourea bond.

The metal chelator is capable of chelating a radioisotope or radionuclide. In some embodiments, the radioisotope or radionuclide is cytotoxic, such as cytotoxic to cancer cells.

The present invention provides for a bioconjugated metal chelator comprising the metal chelator of the present invention. In some embodiments, the metal conjugate is linked to a targeting vector thiourea bond.

In some embodiments, the biological targeting vector is an antibody or a peptide that would target a tissue of interest. In some embodiments, murine antibody, human antibody, humanized antibody, chimeric antibody, or a fragment thereof. In some embodiments, the fragment of an antibody is a Fab, Fv, scFv, diabody, or bispecific scFv. In some embodiments, the antibody is Trastuzumab which targets a breast cancer cell expressing ERBB2. In some embodiments, the peptide is prostate specific membrane antigen (PSMA), which targets a prostate cancer cell. The following table shows monoclonal antibody and the cancer cell which they target:

TABLE 5
Monoclonal antibody and the cancer
cell/molecule which they target.

| Monoclonal Antibody | Cancer Targeted | Target Molecule |
|---|---|---|
| Alemtuzumab | CLL | CD52 |
| Bevacizumab | Colorectal, lung cancer | VEGF |
| Cetuximab | Colorectal cancer | EGFR |
| Gemtuzumab | AML | CD33 |
| Ibritumomab tiuxetan | NHL | CD20 |
| Ofatumumab | CLL | CD20 |
| Panitumumab | Colorectal cancer | EGFR |
| Rituximab | NHL, CLL | CD20 |
| Tositumomab | NHL | CD20 |
| Trastuzumab | Breast cancer | HER-2/neu |

Abbreviations: ADCC, Antibody-dependent cell-mediated cytotoxicity; AML, acute myelogenous leukemia; CLL, chronic lymphocytic leukemia; EGFR, epidermal growth factor receptor; IgG, immunoglobulin G; NHL, non-Hodgkin lymphoma; VEGF, vascular endothelial growth factor.

The present invention provides for a bioconjugated metal chelator comprising metal chelator of the present invention bound to a biological targeting vector (BTV). In some embodiments, the metal chelator is bound to the BTV via a

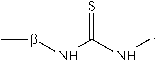

In some embodiments, a radioisotope or radionuclide is bound to the metal chelator. In some embodiments, a radioisotope or radionuclide is a radioisotope or radionuclide described herein, or a mixture thereof. In some embodiments, a radioisotope or radionuclide is Thorium-227 and Zirconium-89, or a mixture thereof. In some embodiments, the metal chelator is DOTHOPO or MeDOTHOPO.

The present invention provides for all precursor and intermediate compounds described herein.

The present invention provides for the making of the metal chelator, and bioconjugate thereof, as described herein. FIG. 7 show the steps for synthesizing a biofunctional metal chelator whereby the -β-N═C═S is bound to the

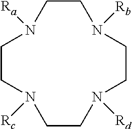

structure first, followed by the addition of Rₐ, R_{b}, R_{c}, and R_{d} using the conditions described herein. FIGS. 8 and 9 show the steps for synthesizing the bioconjugated metal chelator for any biological targeting vector or antibody. The bifunctional metal chelator is bound to the biological targeting vector using the following conditions: aqueous buffer, about pH 8.8 to 9.0, about 37° C., and/or about 30 minutes. The radioisotope or radionuclide is bound to the metal chelator using the following conditions: aqueous buffer, about pH 6.8 to 7.5, about room temperature or 25° C., and/or about 60 minutes.

The present invention provides for a method for constructing a vector of the present invention, the method comprising: introducing the ORF of the hybrid sugar transporter or sugar transporter of the present invention into a vector to produce the sugar transporter of the present invention.

The present invention provides for a method for treating a subject in need of such treatment, the method comprising: administering a therapeutically effective amount of a pharmaceutical composition comprising (i) the bioconjugated metal chelator of the present invention, and (ii) optionally a pharmaceutically acceptable salt, to the subject; wherein the BTV of the bioconjugated metal chelator targets a cancer cell in the subject. In some embodiments, the subject is a human patient, such as a human patient suffering from a cancer.

The metal chelator of the present invention can be used to manufacture and use of pharmaceutical composition, and methods of use thereof, or the binding or separating of industrial or clinical heavy metals or metal ions, using the method steps and components described in the PCT International Patent Application Nos. PCT/US2027/030628, PCT/US2017/050121, and PCT/US2017/048934; U.S. Pat. No. 11,684,614; and, U.S. Patent Application Publication Nos. 2019/0183868 and 2022/0152003, which are incorporated by reference.

The present invention provides for a method for treating a subject for a heavy metal exposure, the method comprising administering a therapeutically effective amount of a pharmaceutical composition comprising the metal chelator to a subject that has an excess amount of one or more of radioisotope or radionuclide, wherein administering results in decorporating, clearing or reducing the amount radioisotope or radionuclide from the subject.

The present invention provides for a method for the prophylactic treatment of a subject for metal exposure, the method comprising administering a therapeutically effective amount of a pharmaceutical composition comprising the metal chelator to a subject.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. X-ray crystal structures of (top) DOTHOPO and (bottom) MeDOTHOPO. Thermal ellipsoids are drawn at the 50% probability level. Outer-sphere solvent molecules and hydrogen atoms bound to carbon atoms are omitted for clarity.

FIG. 2. (a) Spectral changes of solutions containing DOTHOPO and Zr⁴⁺ (c_{L}=c_{M}=2×10⁻⁵ M) over the pH range of 2.4-11.6 (25° C., I=0.1 M KCl). Arrows indicate the growth or disappearance of peaks. (b) Data fitting and species distribution over the titration pH range.

FIG. 3. (a) Spectral changes of solutions containing DOTHOPO and Th⁴⁺ (c_{L}=c_{M}=1.5×10⁻⁵ M) over the pH range of 2.4-12.4 (25° C., I=0.1 M KCl). Arrows indicate the growth or disappearance of peaks. Spectra are corrected for dilution. (b) Data fitting and species distribution over the titration pH range.

FIG. 4. (a,b) Absorbance vs time plots at 669 nm showing the displacement of Arsenazo III from Th-dye complex upon addition of (a) 15 equiv. DOTHOPO and (b) 15 equiv. MeDOTHOPO at t=0 (5 μM Th⁴⁺, 25 μM Arsenazo III, 75 μM ligand, 50 mM HEPES, pH 7.4). (c) Observed first-order rate constants calculated at varying concentrations of ligand relative to Th⁴⁺ showing a lack of dependence on ligand concentration.

FIG. 5. Concentration-dependent radiolabeling plot of (a) DOTA, L₁, DOTHOPO, and MeDOTHOPO with [²²⁷Th]Th⁴⁺ (0.25 μCi, 9.2 kBq) and (b) L₁, DOTHOPO, and MeDOTHOPO with [⁸⁹Zr]Zr⁴⁺ (5 μCi, 185 MBq). All radiolabeling reactions were performed in citrate buffer (0.25 M, pH 5) at 25° C. for 30 min. Results are tabulated in the Supporting Information.

FIG. 6. Serum stability of the (a) [²²⁷Th]Th⁴⁺ and (b) [⁸⁹Zr]Zr⁴⁺ complexes of DOTHOPO and MeDOTHOPO. Results are tabulated in the Supporting Information.

FIG. 7. Synthesis of the bifunctional DOTHOPO. NCS is isothiocyanate.

FIG. 8. Synthesis of a bioconjugated metal chelator comprising the biological targeting vector (∇) conjugated with a metal chelator of the present invention (the circle with a missing quadrant) which chelates a radioisotope (•).

FIG. 9. Synthesis of a bioconjugated metal chelator comprising an antibody (Y-shaped structure) conjugated with a metal chelator of the present invention (the circle with a missing quadrant) which chelates a radioisotope (•).

FIG. 10. Chemical structures of chelators discussed and/or explored in this work.

FIG. 11. Synthesis of ligands described in this report.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Chelators are molecules that can bind metals. Chelators can include organic molecules that covalently bond with a metal. As used herein, a covalent bond describes the sharing of one or more pairs of electrons between atoms. In some instances, chelators are agents that bind to metal.

In some embodiments, chelators can include a number of metal-coordinating atoms that bond with a metal. The metal-coordinating atoms can bond with metals having cations with a +1 charge. The metal-coordinating atoms can also bond with metals having cations with a +2 charge. Additionally, the metal-coordinating atoms can bond with metals having cations with a +3 charge. Further, the metal-coordinating atoms can bond with metals having cations with a +4 charge.

In some embodiments, the metal-coordinating atoms of the chelators described herein can be included in one or more functional groups of the chelators. In some examples, the metal-coordinating atoms of the chelators can be included in one or more catecholate (CAM) groups. A CAM group can include at least a phenyl ring substituted by hydroxyl groups on adjacent carbon atoms.

In some embodiments, the metal-coordinating atoms of the chelators can be included in one or more hydroxamate (HA) groups.

In some embodiments, the metal-coordinating atoms of the chelators can be included in one or more hydroxypyridinone (HOPO) groups. In some embodiments, the HOPO group can include a pyridinone ring substituted by a hydroxyl group on the N atom. In some embodiments, the HOPO group can include a 1,2-HOPO group.

The metal-coordinating atoms of the chelators can be included in combinations of two or more of one or more CAM groups, one or more HA groups, or one or more HOPO groups. In illustrative examples, the metal-coordinating atoms of the chelators can be included in one or more CAM groups and one or more HA groups. In other illustrative examples, the metal-coordinating atoms of the chelators can be included in one or more CAM groups and one or more HOPO groups. In additional illustrative examples, the metal-coordinating atoms of the chelators can be included in one or more HA groups and one or more HOPO groups. In further illustrative examples, the metal-coordinating atoms of the chelators can be included in one or more HA groups, one or more CAM groups, and one or more HOPO groups.

In some embodiments, the chelator is any one or more of the chelators described herein.

Examples of radioisotopes useful include ²²⁵Ac, ²²⁶Ac, ²²⁸Ac, ¹⁰⁵Ag, ¹⁰⁶mAg, ¹¹⁰mAg, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As, ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ¹⁹⁶m²Au, ¹⁹⁸Au, ¹⁹⁸mAu, ¹⁹⁹Au, ²⁰⁰mAu, ¹²⁸Ba, ¹³¹Ba, ¹³³mBa, ¹³⁵mBa, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk, ²⁴⁵Bk, ²⁴⁶Bk, ²⁴⁸mBk, ²⁵⁰Bk, ⁷⁶Br, ⁷⁷Br, ⁸⁰mBr, ⁸²Br, ¹¹C, ¹⁴C, ⁴⁵Ca, ⁴⁷Ca, ¹⁰⁷Cd, ¹¹⁵Cd, ¹¹⁵mCd, ¹¹⁷mCd, ¹³²Ce, ¹³³mCe, ¹³⁴Ce, ¹³⁵Ce, ¹³⁷Ce, ¹³⁷mCe, ¹³⁹Ce, ¹⁴¹Ce, ¹⁴³Ce, ¹⁴⁴Ce, ²⁴⁶Cf, ²⁴⁷Cf, ²⁵³Cf, ²⁵⁴Cf, ²⁴⁰ Cm, ²⁴¹Cm, ²⁴²Cm, ²⁵²Cm, ⁵⁵Co, ⁵⁶Co, ⁵⁷Co, ⁵⁸Co, ⁵⁸mCo, ⁶⁰Co, ⁴⁸Cr, ⁵¹Cr, ¹²⁷Cs, ¹²⁹Cs, ¹³¹Cs, ¹³²Cs, ¹³⁶Cs, ¹³⁷Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er, ¹⁶¹Er, ¹⁶⁵Er, ¹⁶⁹Er, ¹⁷¹Er, ¹⁷²Er, ²⁵⁰Es, ²⁵¹Es, ²⁵³Es, ²⁵⁴Es, ²⁵⁴mEs, ²⁵⁵Es, ²⁵⁶mEs, ¹⁴⁵Eu, ¹⁴⁶Eu, ¹⁴⁷Eu, ¹⁴⁸Eu, ¹⁴⁹Eu, ¹⁵⁰mEu, ¹⁵²mEu, ¹⁵⁶Eu, ¹⁵⁷Eu, ⁵²Fe, ⁵⁹Fe, ²⁵¹Fm, ²⁵²Fm, ²⁵³Fm, ²⁵⁴Fm, ²⁵⁵Fm, ²⁵⁷Fm, ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge, ¹⁷⁰Hf, ¹⁷¹Hf, ¹⁷³ Hf, ¹⁷⁵Hf, ¹⁷⁹m²Hf, ¹⁸⁰mHf, ¹⁸¹Hf, ¹⁸⁴Hf, ¹⁹²Hg, ¹⁹³Hg, ¹⁹³mHg, ¹⁹⁵Hg, ¹⁹⁵mHg, ¹⁹⁷Hg, ¹⁹⁷mHg, ²⁰³Hg, ¹⁶⁰mHo, ¹⁶⁶Ho, ¹⁶⁷Ho, ¹²³I, ¹²⁴I, ¹²⁹I, ¹³⁰I, ¹³²I, ¹³³I, ¹³⁵I, ¹⁰⁹In, ¹¹⁰In, ¹¹¹In, ¹¹⁴mIn, ¹¹⁵mIn, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ¹⁹⁰m²Ir, ¹⁹²Ir, ¹⁹³mIr, ¹⁹⁴Ir, ¹⁹⁴m²Ir, ¹⁹⁵mIr, ⁴²K, ⁴³K, ⁷⁶Kr, ⁷⁹Kr, ⁸¹mKr, ⁸⁵mKr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ¹⁷⁴mLu, ¹⁷⁶mLu, ¹⁷⁷Lu, ¹⁷⁷mLu, ¹⁷⁹Lu, ²⁵⁷Md, ²⁵⁸Md, ²⁶⁰Md, ²⁸Mg, ⁵²Mn, ⁹⁰Mo, ⁹³mMo, ⁹⁹Mo, ¹³N, ²⁴Na, ⁹⁰Nb, ⁹¹mNb, ⁹²mNb, ⁹⁵Nb, ⁹⁵mNb, ⁹⁶Nb, ¹³⁸Nd, ¹³⁹mNd, ¹⁴⁰Nd, ¹⁴⁷Nd, ⁵⁶Ni, ⁵⁷Ni, ⁶⁶Ni, ²³⁴Np, ²³⁶mNp, ²³⁸Np, ²³⁹Np, ¹⁵O, ¹⁸²Os, ¹⁸³Os, ¹⁸³mOs, ¹⁸⁵Os, ¹⁸⁹mOs, ¹⁹¹Os, ¹⁹¹mOs, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd, ¹¹¹mPd, ¹¹²Pd, ¹⁴³Pm, ¹⁴⁸Pm, ¹⁴⁸mPm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁸⁹Pt, ¹⁹¹Pt, ¹⁹³mPt, ¹⁹⁵mPt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu, ²²³Ra, ²²⁴Ra, ²²⁵Ra, ⁸¹Rb, ⁸²Rb, ⁸²mRb, ⁸³Rb, ⁸⁴Rb, 86Rb, ¹⁸¹Re, ¹⁸²Re, ¹⁸²mRe, ¹⁸³Re, ¹⁸⁴Re, ¹⁸⁴mRe, ¹⁸⁶Re, ¹⁸⁸Re, ¹⁸⁹Re, ¹⁹⁰mRe, ⁹⁹Rh, ⁹⁹mRh, ¹⁰⁰Rh, ¹⁰¹mRh, ¹⁰²Rh, ¹⁰³mRh, ¹⁰⁵Rh, ²¹¹Rn, ²²²Rn, ⁹⁷Ru, ¹⁰³Ru, ¹⁰⁵Ru, ³⁵S, ¹¹⁸mSb, ¹¹⁹Sb, ¹²⁰Sb, ¹²⁰mSb, ¹²²Sb, ¹²⁴Sb, ¹²⁶Sb, ¹²⁷Sb, ¹²⁸Sb, ¹²⁹Sb, ¹³Sc, ⁴⁴Sc, ⁴⁴mSc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ¹¹⁷mSn, ¹¹⁹mSn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta, ¹⁸³Ta, ¹⁸⁴Ta, ¹⁴⁹Th, ¹⁵⁰Tb, ¹⁵¹Tb, ¹⁵²Tb, ¹⁵³Tb, ¹⁵⁴Tb, ¹⁵⁴mTb, ¹⁵⁴m²Tb, ¹⁵⁵Tb, ¹⁵⁶Tb, ¹⁵⁶mTb, ¹⁵⁶m²Tb, ¹⁶⁰Tb, ¹⁶¹Tb, ⁹⁴Tc, ⁹⁵Tc, ⁹⁵mTc, ⁹⁶Tc, ⁹⁷mTc, ⁹⁹mTc, ¹¹⁸Te, ¹¹⁹Te, ¹¹⁹mTe, ¹²¹Te, ¹²¹mTe, ¹²³mTe, ¹²⁵mTe, ¹²⁷Te, ¹²⁷mTe, ¹²⁹mTe, ¹³¹mTe, ¹³²Te, ²²⁷Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ¹²⁹mXe, ¹³¹mXe, ¹³³Xe, ¹³³mXe, ¹³⁵Xe, ⁸⁵mY, ⁸⁶Y, ⁸⁷Y, ⁸⁷mY, ⁸⁸Y, ⁹⁰Y, ⁹⁰mY, ⁹¹Y, ⁹²Y, ⁹³Y, ¹⁶⁶Yb, ¹⁶⁹Yb, ¹⁷⁵Yb, ⁶²Zn, ⁶⁵Zn, ⁶⁹mZn, ⁷¹mZn, ⁷²Zn, ⁸⁶Zr, ⁸⁸Zr, ⁸⁹Zr, ⁹⁵Zr, and ⁹⁷Zr.

It can be helpful to classify cytotoxic radionuclides into groups, for example, metals (e.g., ⁹⁰Y, ⁶⁷Cu, ²¹³Bi, ²¹²Bi), and transitional elements (e.g., ¹⁸⁶Re). Further, examples of pure β-emitters include ⁶⁷Cu and ⁹⁰Y; and examples of α-emitters include ²¹³Bi. β-emitters that emit γ-radiation include ¹⁷⁷Lu and ¹⁸⁶Re, while Auger emitters and radionuclides that decay by internal conversion include ⁶⁷Ga.

As will be appreciated by one of ordinary skill in the art, more than one radioisotope may be chosen and used, for example, in particular nuclear medicine indications. Thus, embodiments can include a single species of radioisotope, two species of radioisotopes, or a population of a plurality of species of radioisotopes combined in various proportions. In this manner the useful properties of different radioisotopes can be combined. For example, a single radioisotope decays at a defined exponential rate. By combining radioisotopes of different half-lives, it is possible to create a new decay rate.

In particular embodiments, biological targeting vectors can be derived from whole proteins or protein fragments with an affinity for particular tissues and/or cell types of interest. In particular embodiments, biological targeting vectors can be derived from whole antibodies or binding fragments of an antibody, e.g., Fv, Fab, Fab′, F(ab′)₂, Fc, and single chain Fv fragments (scFvs) or any biologically-effective fragments of an immunoglobulin that bind specifically to, for example, a cancer antigen epitope. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Biological targeting vectors from human origin or humanized antibodies have lowered or no immunogenicity in humans and have a lower number of non-immunogenic epitopes compared to non-human antibodies. Antibodies and their fragments will generally be selected to have a reduced level or no antigenicity in human subjects. Biological targeting vectors can particularly include any peptide that specifically binds a selected unwanted cell epitope. Sources of biological targeting vectors include antibody variable regions from various species (which can be in the form of antibodies, sFvs, scFvs, Fabs, scFv-based grababody, or soluble VH domain or domain antibodies). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

Phage display libraries of partially or fully synthetic antibodies are available and can be screened for an antibody or fragment thereof that can bind a selected epitope. For example, biological targeting vectors may be identified by screening a Fab phage library for Fab fragments that specifically bind to a target of interest (see Hoet et al., Nat. Biotechnol. 23:344, 2005). Phage display libraries of human antibodies are also available. Additionally, traditional strategies for hybridoma development using a target of interest as an immunogen in convenient systems (e.g., mice, HuMAb Mouse®, TC Mouse™, KM-Mouse®, llamas, chicken, rats, hamsters, rabbits, etc.) can be used to develop biological targeting vectors. In particular embodiments, antibodies specifically bind to selected epitopes expressed by targeted cells and do not cross react with nonspecific components or unrelated targets. Once identified, the amino acid sequence or polynucleotide sequence coding for the antibody can be isolated and/or determined.

An alternative source of biological targeting vectors includes sequences that encode random peptide libraries or sequences that encode an engineered diversity of amino acids in loop regions of alternative non-antibody scaffolds, such as scTCR (see, e.g., Lake et al., Int. Immunol. 11:745, 1999; Maynard et al., J. Immunol. Methods 306:51, 2005; U.S. Pat. No. 8,361,794), mAb2 or Fcab™ (see, e.g., PCT Patent Application Publication Nos. WO 2007/098934; WO 2006/072620), affibody, avimers, fynomers, cytotoxic T-lymphocyte associated protein-4 (Weidle et al., Cancer Gen. Proteo. 10:155, 2013), or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

An “antibody fragment” denotes a portion of a complete or full length antibody that retains the ability to bind to an epitope. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the VL and VH domains of a single arm of an antibody. Although the two domains of the Fv fragment, VL and VH, are coded by separate genes, they can be joined, using, for example, recombinant methods, by a synthetic linker that enables them to be made as a single protein chain in which the VL and VH regions pair to form monovalent molecules (single chain Fv (scFv)). For additional information regarding Fv and scFv, see e.g., Bird, et al., Science 242 (1988) 423-426; Huston, et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. Nos. 5,571,894; and 5,587,458.

A Fab fragment is a monovalent antibody fragment including VL, VH, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90 (1993) 6444-6448. Dual affinity retargeting antibodies (DART™; based on the diabody format but featuring a C-terminal disulfide bridge for additional stabilization (Moore et al., Blood 117, 4542-51 (2011))) can also be used. Antibody fragments can also include isolated CDRs. For a review of antibody fragments, see Hudson, et al., Nat. Med. 9 (2003) 129-134.

Antibody fragments can be made by various techniques, including proteolytic digestion of an intact antibody as well as production by recombinant host-cells (e.g. E. coli or phage), as described herein. Antibody fragments can be screened for their binding properties in the same manner as intact antibodies.

In particular embodiments, biological targeting vectors can also include a natural receptor or ligand for an epitope. For example, if a target for binding includes PD-L1, the binding domain can include PD-1 (including, e.g., a PD-1/antiCD3 fusion). One example of a receptor fusion for binding is Enbrel® (Immunex). Natural receptors or ligands can also be modified to enhance binding. For example, betalacept is a modified version of abatacept.

Binding can also be enhanced through increasing avidity. Any screening method known in the art can be used to identify increased avidity to an antigen epitope.

As used herein, an epitope denotes the binding site on a protein target bound by a corresponding biological targeting vector. The biological targeting vectors either binds to a linear epitope, (e.g., an epitope including a stretch of 5 to 12 consecutive amino acids), or the biological targeting vectors binds to a three-dimensional structure formed by the spatial arrangement of several short stretches of the protein target. Three-dimensional epitopes recognized by a biological targeting vectors, e.g. by the epitope recognition site or paratope of an antibody or antibody fragment, can be thought of as three-dimensional surface features of an epitope molecule. These features fit precisely (in) to the corresponding binding site of the biological targeting vectors and thereby binding between the biological targeting vectors and its target protein is facilitated.

“Bind” means that the biological targeting vectors associates with its target epitope with a dissociation constant (1(D) of 10⁻⁸ M or less, in one embodiment of from 10⁻⁵ M to 10⁻¹³ M, in one embodiment of from 10⁻⁵M to 10⁻¹⁰ M, in one embodiment of from 10⁻⁵ M to 10⁻⁷ M, in one embodiment of from 10⁻⁸ M to 10⁻¹³ M, or in one embodiment of from 10⁻⁹ M to 10⁻¹³ M. The term can be further used to indicate that the biological targeting vectors does not bind to other biomolecules present, (e.g., it binds to other biomolecules with a dissociation constant (KD) of 10⁻⁴ M or more, in one embodiment of from 10⁻⁴ M to 1 M.

Methods of Synthesizing Chelators. In some embodiments, compositions of chelators described herein can be synthesized using techniques described herein.

Methods of Making Radionuclides. Radioisotopes can be obtained in solution in water or other polar fluid in elemental form (i.e., uncharged) or ionic form. As appreciated by the skilled artisan, when in ionic form, radioisotopes may occur in various different valence states, as anions, or as cations, depending upon the particular radioisotope being considered.

Methods of Charging Chelators with Radionuclides. In particular embodiments, chelators can be charged with radionuclides by contacting the chelators with metallic radioisotopes and allowing complexes between the two molecules to form.

A prodrug includes an active ingredient which is converted into a therapeutically active or more therapeutically active compound after administration, such as by cleavage of a protein.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt. Suitable pharmaceutically acceptable acid addition salts can be prepared from an inorganic acid or an organic acid. Examples of inorganic acids include hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids. Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

The term analog (also structural analog or chemical analog) is used to refer to a compound that is structurally similar to another compound but differs with respect to a certain component, such as an atom, a functional group, or a substructure. The term derivative refers to a compound that is obtained from a similar compound or a precursor compound by a chemical reaction. As used herein, analogs and derivatives retain the therapeutic effectiveness of the parent compound (i.e., there is no statistically significant difference in therapeutic activity according to an imaging assay or assessment of clinical improvement) or have improved therapeutic effectiveness as defined elsewhere herein.

Active ingredients are formulated into compositions for administration to subjects. Compositions include at least one active ingredient and at least one pharmaceutically acceptable carrier. In particular embodiments, compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent for use as a pharmaceutically acceptable carrier is EDTA. Other chelating agents disclosed herein may also be used.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredient or helps to prevent denaturation or adherence to the container wall. Typical stabilizers can include polyhydric sugar alcohols; amino acids, such as R, K, G, Q, N, H, A, ornithine, L-leucine, 2-F, E, and T; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on active ingredient weight.

In particular embodiments, the compositions disclosed herein can be formulated for administration by injection (e.g., intravenous injection). Compositions can also be formulated for administration by, for example, inhalation, infusion, perfusion, lavage, or ingestion. The compositions disclosed herein can further be formulated for intradermal, intraarterial, intranodal, intralymphatic, intraperitoneal, intralesional, intraprostatic, intravaginal, intrarectal, topical, intrathecal, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intramuscular, intravesicular, and/or subcutaneous injection.

For injection, compositions can be formulated as aqueous solutions, such as in buffers including Hanks' solution, Ringer's solution, or physiological saline. The aqueous solutions can contain formulatory agents such as suspending, stabilizing, and/or dispersing agents. Alternatively, the formulation can be in lyophilized and/or powder form for constitution with a suitable vehicle, e.g., sterile pyrogen-free water, before use. Particular embodiments are formulated for intravenous or intramuscular administration.

For oral administration, the compositions can be formulated as tablets, pills, dragees, capsules, liquids, gels, syrups, slurries, suspensions and the like. Compositions can be formulated as an aerosol for inhalation. In one embodiment, the aerosol is provided as part of an anhydrous, liquid or dry powder inhaler. Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts. Additionally, compositions can be formulated as sustained-release systems utilizing semipermeable matrices of solid polymers containing at least one active ingredient.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Kits. Also disclosed herein are kits including one or more containers including one or more of the active ingredients, compositions, chelators, and/or radionuclides described herein. In various embodiments, the kits may include one or more containers containing one or more portions of active ingredients and/or compositions to be used in combination with other portions of the active ingredients and/or compositions described herein. Associated with such container(s) can be a notice in the form prescribed by a governmental agency regulating the manufacture, use, or sale of pharmaceuticals or biological products, which notice reflects approval by the agency of manufacture, use, or sale for human administration.

Optionally, the kits described herein further include instructions for using the kit in the methods disclosed herein. In various embodiments, the kit may include instructions regarding preparation of the active ingredients and/or compositions for administration; administration of the active ingredients and/or compositions; appropriate reference levels to interpret results associated with using the kit; proper disposal of the related waste; and the like. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. The instructions may be in English and/or in any national or regional language. In various embodiments, possible side effects and contraindications to further use of components of the kit based on a subject's symptoms can be included.

In various embodiments, the kits described herein include some or all of the necessary medical supplies needed to use the kit effectively, thereby eliminating the need to locate and gather such medical supplies. Such medical supplies can include syringes, ampules, tubing, facemasks, protective clothing, a needleless fluid transfer device, an injection cap, sponges, sterile adhesive strips, Chloraprep, gloves, and the like. Variations in contents of any of the kits described herein can be made. Particular kits provide materials to administer compositions through intravenous administration.

Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with therapeutic compositions disclosed herein. Treating subjects includes delivering therapeutically effective amounts. Therapeutically effective amounts include those that provide effective amounts and therapeutic treatments.

An “effective amount” is the amount of a composition necessary to result in a desired physiological change in the subject. Effective amounts are often administered for research purposes. Effective amounts disclosed herein can cause a statistically-significant effect in an animal model assessing a use of nuclear medicine.

A “therapeutic treatment” can include a treatment administered to a subject in need of imaging. The subject can be in need of imaging to aid in diagnosis; to locate a position for a therapeutic intervention; to assess the functioning of a body part; and/or to assess the presence or absence of a condition. The effectiveness of a therapeutic imaging treatment can be confirmed based on the capture of an image sufficient for its intended purpose.

Exemplary types of imaging that utilize nuclear medicine include: positron emission tomography (PET), single photon emission computed tomography, radioisotope renography, and scintigraphy.

A “therapeutic treatment” can also include a treatment administered to a subject with a condition. The therapeutic treatment reduces, controls, or eliminates the condition or a symptom associated with the condition. Conditions treated with nuclear medicine include those associated with the proliferation of unwanted cells.

In particular embodiments, therapeutic treatments reduce cellular proliferation. Cellular proliferation refers to the process of cellular division, either through mitosis or meiosis, whereby increased cell numbers result. In particular embodiments, therapeutic treatments reduce cellular growth. Cellular growth refers both to an increase in cell mass or size, as well as cellular physiological processes necessary to support a cell's life.

Particular conditions that can be treated include various cancers, thyroid diseases (e.g., hyperthyroidism or thyrotoxicosis), blood disorders (e.g., Polycythemia vera, an excess of red blood cells produced in the bone marrow), and cellular proliferation in blood vessels following balloon angioplasty and/or stent placement (known as restenosis).

The effectiveness of a therapeutic treatment can be confirmed based on a beneficial change related to the condition following the treatment.

In the context of cancers, therapeutic treatments can decrease the number of cancer cells, decrease the number of metastases, decrease tumor volume, increase life expectancy, induce chemo- or radiosensitivity in cancer cells, inhibit angiogenesis near cancer cells, inhibit cancer cell proliferation, inhibit tumor growth, prevent or reduce metastases, prolong a subject's life, reduce cancer-associated pain, and/or reduce relapse or re-occurrence of cancer following treatment. In particular embodiments, therapeutic treatments reduce, delay, or prevent further metastasis from occurring.

As indicated previously, particular uses of the chelating platforms disclosed herein include in imaging and treatment in the same subject.

The actual dose amount administered to a particular subject can be determined by a physician, veterinarian, or researcher taking into account parameters such as physical and physiological factors including body weight; severity of condition; previous or concurrent therapeutic interventions; idiopathy of the subject; and route of administration.

In particular embodiments, the total dose of absorbed radiation may include 10-3 grays (Gy), 10-2 Gy, 10-1 Gy, 1 Gy, 5 Gy, 10 Gy, 25 Gy, 50 Gy, 75 Gy, 100 Gy, 200 Gy, 300 Gy, 400 Gy, 500 Gy, 600 Gy, 700 Gy, 800 Gy, 900 Gy, or 1000 Gy.

Doses of absorbed radiation can be achieved by delivering an appropriate amount of a composition. Exemplary amounts of compositions can include 0.05 mg/kg to 5.0 mg/kg administered to a subject per day in one or more doses. For certain indications, the total daily dose can be 0.05 mg/kg to 3.0 mg/kg administered intravenously to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of composition using 60-minute QD, BID, or TID intravenous infusion dosing. Additional useful doses can often range from 0.1 to 5 μg/kg or from 0.5 to 1 μg/kg. In other examples, a dose can include 1 μg/kg, 20 μg/kg, 40 μg/kg, 60 μg/kg, 80 μg/kg, 100 μg/kg, 200 μg/kg, 350 μg/kg, 500 μg/kg, 700 μg/kg, 0.1 to 5 mg/kg, or from 0.5 to 1 mg/kg. In other examples, a dose can include 1 mg/kg, 10 mg/kg, 20 mg/kg, 40 mg/kg, 60 mg/kg, 80 mg/kg, 100 mg/kg, 200 mg/kg, 400 mg/kg, 500 mg/kg, 700 mg/kg, 750 mg/kg, 1000 mg/kg, or more.

Therapeutically effective amounts can be achieved by administering single or multiple doses during the course of an imaging or treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly).

References cited herein:
(1) Guerra Liberal, F. D. C.; O'Sullivan, J. M.; McMahon, S. J.; Prise, K. M. Targeted Alpha Therapy: Current Clinical Applications. Cancer Biother. Radiopharm. 2020, 35, 404-417.
(2) Du, Y.; Carrio, I.; De Vincentis, G.; Fanti, S.; Ilhan, H.; Mommsen, C.; Nitzsche, E.; Sundram, F.; Vogel, W.; Oyen, W.; Lewington, V. Practical Recommendations for Radium-223 Treatment of Metastatic Castration-Resistant Prostate Cancer. Eur. J. Nucl. Med. Mol. Imaging 2017, 44, 1671-1678.
(3) De Vincentis, G.; Follacchio, G. A.; Frantellizzi, V.; Prelaj, A.; Farcomeni, A.; Giuli, A.; Bianco, V.; Tomao, S. ²²³Ra-Dichloride Therapy in an Elderly Bone Metastatic Castration-Resistant Prostate Cancer Patient: A Case Report Presentation and Comparison with Existing Literature. Aging Clin. Exp. Res. 2018, 30, 677-680.
(4) Price, E. W.; Orvig, C. Matching Chelators to Radiometals for Radiopharmaceuticals. Chem. Soc. Rev. 2014, 43. 260-290.
(5) Kostelnik, T. I.; Orvig, C. Radioactive Main Group and Rare Earth Metals for Imaging and Therapy. Chem. Rev. 2019, 119, 902-956.
(6) Abou, D. S.; Thiele, N. A.; Gutsche, N. T.; Villmer, A.; Zhang, H.; Woods, J. J.; Baidoo, K. E.; Escorcia, F. E.: Wilson, J. J.; Thorek, D. L. J. Towards the Stable Chelation of Radium for Biomedical Applications with an 18-Membered Macrocyclic Ligand. Chem. Sci. 2021, 12, 3733-3742.
(7) Ivanov, A. S.; Simms, M. E.; Bryantsev, V. S.; Benny, P. D.; Griswold, J. R.; Delmau, L. H.; Thiele, N. A. Elucidating the Coordination Chemistry of the Radium Ion for Targeted Alpha Therapy. Chem. Commun. 2022, 58, 9938-9941.
(8) Hagemann, U. B.: Wickstroem, K.; Hammer, S.; Bjerke, R. M.; Zitzmann-Kolbe, S.; Ryan, O. B.; Karlsson, J.; Scholz, A.; Hennekes, H.; Mumberg, D.; Cuthbertson, A. S. Advances in Precision Oncology: Targeted Thorium-227 Conjugates As a New Modality in Targeted Alpha Therapy. Cancer Biother. Radiopharm. 2020, 35, 497-510.
(9) Grieve, M. L.; Paterson, B. M. The Evolving Coordination Chemistry of Radiometals for Targeted Alpha Therapy. Aust. J. Chem. 2022, 75. 65-88.
(10) Birnbaum, E. R.; Fassbender, M. E.; Ferrier, M. G.; John, K. D.; Mastren, T. Actinides in Medicine. Encycl. Inorg. Bioinorg. Chem. 2018, eibc2563.
(11) Radchenko, V.; Morgenstern, A.; Jalilian, A. R.; Ramogida, C. F.; Cutler, C.; Duchemin, C.; Hoehr, C.; Haddad, F.; Bruchertseifer, F.; Gausemel, H.; Yang, H.; Osso, J. A.; Washiyama, K.; Czerwinski, K.; Leufgen, K.; Pruszyński, M.; Valzdorf, O.; Causey, P.; Schaffer, P.; Perron, R.; Maxim, S.; Wilbur, D. S.; Stora, T.; Li, Y. Production and Supply of α-Particle-Emitting Radionuclides for Targeted α-Therapy. J. Nucl. Med. 2021, 62, 1495-1503.
(12) Raju, M. R.; Eisen, Y.; Carpenter, S.; Inkret, W. C. Radiobiology of a Particles: III. Cell Inactivation by α-Particle Traversals of the Cell Nucleus. Radiat. Res. 1991, 128, 204-209
(13) Behr, T. M.; Béhé, M.; Stabin, M. G.; Wehrmann, E.; Apostolidis, C.; Molinet, R.; Strutz, F.; Fayyazi, A.; Wieland, E.; Gratz, S.; Koch, L.; Goldenberg, D. M.; Becker, W. High-Linear Energy Transfer (LET) a versus Low-LET B Emitters in Radioimmunotherapy of Solid Tumors: Therapeutic Efficacy and Dose-Limiting Toxicity of ²¹³Bi-versus ⁹⁰Y-Labeled CO17-1a Fab′ Fragments in a Human Colonic Cancer Model. Cancer Res. 1999, 59, 2635-2643.
(14) Benabdallah, N.; Scheve, W.; Dunn, N.; Silvestros, D.; Schelker, P.; Abou, D.; Jammalamadaka, U.; Laforest, R.; Li, Z.; Liu, J.; Ballard, D. H.; Maughan, N. M.; Gay, H.; Baumann, B. C.; Hobbs, R. F.; Rogers, B.; Iravani, A.; Jha. A. K.; Dehdashti, F.; Thorek, D. L. J. Practical Considerations for Quantitative Clinical SPECT/CT Imaging of Alpha Particle Emitting Radioisotopes. Theranostics 2021, 11, 9721-9737.
(15) Murray, I.; Rojas, B.; Gear, J.; Callister, R.; Cleton, A.; Flux, G. D. Quantitative Dual-Isotope Planar Imaging of Thorium-227 and Radium-223 Using Defined Energy Windows. Cancer Biother. Radiopharm. 2020, 35, 530-539.
(16) Z. Soderquist, C.; K. McNamara, B.; R. Fisher, D. Production of High-Purity Radium-223 from Legacy Actinium-Beryllium Neutron Sources. Curr. Radiopharm. 2012, 5, 244-252.
(17) Eychenne, R.; Chérel, M.; Haddad, F.; Guérard, F.; Gestin, J.-F. Overview of the Most Promising Radionuclides for Targeted Alpha Therapy: The “Hopeful Eight.” Pharmaceutics 2021. 13, 906.
(18) Morgenstern, A.; Abbas, K.; Bruchertseifer, F.; Apostolidis, C. Production of Alpha Emitters for Targeted Alpha Therapy. Curr. Radiopharm. 2008, 1, 135-143.
(19) Day, R. A.; Stoughton, R. W. Chemistry of Thorium in Aqueous Solutions. I. Some Organic and Inorganic Complexes. J. Am. Chem. Soc. 1950, 72, 5662-5666.
(20) Waggener, W. C.; Stoughton, R. W. Chemistry of Thorium in Aqueous Solutions. II. Chloride Complexing as A Function of Ionic Strength. J. Phys. Chem. 1952, 56, 1-5.
(21) Daly, S. R.; Piccoli, P. M. B.; Schultz, A. J.; Todorova, T. K.; Gagliardi, L.; Girolami, G. S. Synthesis and Properties of a Fifteen-Coordinate Complex: The Thorium Aminodiboranate [Th(H₃BNMe₂BH₃)₄]. Angew. Chem. Int. Ed 2010, 49, 3379-3381.
(22) Tutson, C. D.; Gorden, A. E. V. Thorium Coordination: A Comprehensive Review Based on Coordination Number. Coord. Chem. Rev. 2017, 333, 27-43.
(23) Torapava, N.; Persson, 1.; Eriksson, L.; Lundberg, D. Hydration and Hydrolysis of Thorium (IV) in Aqueous Solution and the Structures of Two Crystalline Thorium (IV) Hydrates. Inorg. Chem. 2009, 48, 11712-11723.
(24) Knope, K. E.; Soderholm, L. Solution and Solid-State Structural Chemistry of Actinide Hydrates and Their Hydrolysis and Condensation Products. Chem. Rev. 2013, 113, 944-994.
(25) Müller, W. A. Studies on Short-Lived Internal α-Emitters in Mice and Rats Part II. ²²⁷Th. Int. J. Radiat. Biol. 1971, 20, 233-243.
(26) Vidaud, C.; Bourgeois, D.; Meyer, D. Bone as Target Organ for Metals: The Case of f-Elements. Chem. Res. Toxicol. 2012, 25, 1161-1175.
(27) Tamain, C.; Dumas, T.; Hennig, C.; Guilbaud, P. Coordination of Tetravalent Actinides (An=Th^{IV}, U^{IV}, Np^{IV}, Pu^{IV}) with DOTA: From Dimers to Hexamers. Chem.—A Eur. J. 2017, 23, 6864-6875.
(28) Kent, G. T.; Wu, G.; Hayton, T. W. Synthesis and Crystallographic Characterization of the Tetravalent Actinide-DOTA Complexes [An^{IV}(κ⁸-DOTA)(DMSO)](An=Th, U). Inorg. Chem. 2019, 58, 8253-8256.
(29) Dahle, J.; Borrebæk, J.; Melhus, K. B.; Bruland, Ø. S.; Salberg, G.; Olsen, D. R.; Larsen, R. H. Initial Evaluation of ²²⁷Th-p-Benzyl-DOTA-Rituximab for Low-Dose Rate α-Particle Radioimmunotherapy. Nucl. Med. Biol. 2006, 33, 271-279.
(30) Abbas, N.; Heyerdahl, H.; Bruland, Ø. S.; Borrebæk, J.; Nesland, J.; Dahle, J. Experimental α-Particle Radioimmunotherapy of Breast Cancer Using ²²⁷Th-Labeled p-Benzyl-DOTA-Trastuzumab. EJNMMI Res. 2011. 1. 18.
(31) Heyerdahl, H.; Abbas, N.; Brevik, E. M.; Mollatt, C.; Dahle, J. Fractionated Therapy of HER2-Expressing Breast and Ovarian Cancer Xenografts in Mice with Targeted Alpha Emitting ²²⁷Th-DOTA-p-Benzyl-Trastuzumab. PLOS One 2012, 7, e42345.
(32) Staudacher, A. H.; Bezak, E.; Borysenko, A.; Brown, M. P. Targeted α-Therapy Using 227Th-APOMAB and Cross-Fire Antitumour Effects: Preliminary in-Vivo Evaluation. Nucl. Med. Commun. 2014, 35, 1284-1290.
(33) Henriksen, G.; Bruland, Ø. S.; Larsen, R. H. Thorium and Actinium Polyphosphonate Compounds As Bone-Seeking Alpha Particle-Emitting Agents. Anticancer Res. 2004, 24, 101-105.
(34) Washiyama, K.; Amano, R.; Sasaki, J.; Kinuya, S.; Tonami, N.; Shiokawa, Y.; Mitsugashira, T. ²²⁷Th-EDTMP: A Potential Therapeutic Agent for Bone Metastasis. Nucl. Med. Biol. 2004, 31, 901-908.
(35) Pham, T. A.; Xu, J.; Raymond, K. N. A Macrocyclic Chelator with Unprecedented Th⁴⁺ Affinity. J. Am. Chem. Soc. 2014, 136, 9106-9115.
(36) Ferrier, M. G.; Li, Y.; Chyan, M. K.; Wong, R.; Li, L.; Spreckelmeyer, S.; Hamlin, D. K.; Mastren, T.; Fassbender, M. E.; Orvig, C.; Wilbur, D. S. Thorium Chelators for Targeted Alpha Therapy: Rapid Chelation of Thorium-226. J. Label. Compd. Radiopharm. 2020, 63, 502-516.
(37) Labadie, K. P.; Hamlin, D. K.; Kenoyer, A.; Daniel, S. K.; Utria, A. F.; Ludwig, A. D.; Kenerson, H. L.; Li, L.; Sham, J. G.; Chen, D. L.; Orozco, J. J.; Yeung, R. S.; Orvig, C.; Li, Y.; Wilbur, D. S.; Park, J. O. Glypican-3-Targeted ²²⁷Th α-Therapy Reduces Tumor Burden in an Orthotopic Xenograft Murine Model of Hepatocellular Carcinoma. J. Nucl. Med. 2022, 63, 1033-1038
(38) Foster, A.; Nigam, S.; Tatum, D. S.; Raphael, I.; Xu, J.; Kumar, R.; Plakseychuk, E.; Latoche, J. D.; Vincze, S.; Li, B.; Giri, R.; McCarl, L. H.; Edinger, R.; Ak, M.; Peddagangireddy, V.; Foley, L. M.; Hitchens, T. K.; Colen, R. R.; Pollack, I. F.; Panigrahy, A.: Magda, D.; Anderson, C. J.; Edwards, W. B.; Kohanbash, G. Novel Theranostic Agent for PET Imaging and Targeted Radiopharmaceutical Therapy of Tumour-Infiltrating Immune Cells in Glioma. EBioMedicine 2021, 71.
(39) Abou, D. S.; Longtine, M.; Shim, K.; Fears, A.; Unnerstall, R.; Hasson, A.; Benabdallah, N.; Zhang. H.; Mangin, F.; Ozen, S.; Raibaut, L.; Brandès, S.; Meyer, M.; Chambron, J. C.; Tatum, D. S.; Magda, D.; Wahl, R.; Thorek, D. L. J. Comparing Four Chelators of Thorium-227 and Antibody Conjugates for Alpha-Emitting Radioimmunotherapy. J. Nucl. Med. 2022, 63, 2568.
(40) Abou, D. S.; Longtine, M.; Fears, A.; Benabdallah, N.; Unnerstall, R.; Johnston, H.; Shim, K.; Hasson, A.; Zhang, H.; Ulmert, D.; Mangin, F.; Ozen, S.; Raibaut, L.; Brandės, S.; Meyer, M.; Chambron, J.-C.; Tatum, D. S.; Magda, D.; Wahl, R. L.; Thorek, D. L. J. Evaluation of Candidate Theranostics for ²²⁷Th/⁸⁹Zr Paired Radioimmunotherapy of Lymphoma. J. Nucl. Med. 2023.
(41) Carbo-Bague, I.; Li. C.; McNeil, B. L.; Gao, Y.; McDonagh, A. W.; Van de Voorde, M.; Ooms, M.; Kunz, P.; Yang, H.; Radchenko, V.; Schreckenbach, G.; Ramogida, C. F. Comparative Study of a Decadentate Acyclic Chelate, HOPO-O₁₀, and Its Octadentate Analogue, HOPO-O₈, for Radiopharmaceutical Applications. Inorg. Chem. 2023.
(42) Captain, I.; Deblonde, G. J. P.; Rupert, P. B.; An, D. D.; Illy, M. C.; Rostan, E.; Ralston, C. Y.; Strong. R. K.; Abergel, R. J. Engineered Recognition of Tetravalent Zirconium and Thorium by Chelator-Protein Systems: Toward Flexible Radiotherapy and Imaging Platforms. Inorg. Chem. 2016, 55, 11930-11936.
(43) Deri, M. A.; Ponnala, S.; Zeglis, B. M.; Pohl, G.; Dannenberg, J. J.; Lewis, J. S.; Francesconi, L. C. Alternative Chelator for ⁸⁹Zr Radiopharmaceuticals: Radiolabeling and Evaluation of 3,4,3-(LI-1,2-HOPO). J. Med. Chem. 2014, 57, 4849-4860.
(44) Bhupathiraju, N. V. S. D. K.; Younes, A.; Cao, M.; Ali, J.; Cicek, H. T.; Tully, K. M.; Ponnala, S.; Babich, J. W.; Deri, M. A.; Lewis, J. S.; Francesconi, L. C.; Drain, C. M. Improved Synthesis of the Bifunctional Chelator P—SCN-Bn-HOPO. Org. Biomol. Chem. 2019. 17. 6866-6871.
(45) Deri, M. A.; Ponnala, S.; Kozlowski, P.; Burton-Pye, B. P.; Cicek, H. T.; Hu, C.; Lewis, J. S.; Francesconi, L. C. P—SCN-Bn-HOPO: A Superior Bifunctional Chelator for ⁸⁹Zr ImmunoPET. Bioconjug. Chem. 2015, 26, 2579-2591.
(46) Sanders, V.; Pratt, E.; Bhupathiraju, D.; Carter, L.; Ferdous, J.; Marquez-Nostra, B.; Schlyer, D.; Lewis, J. S.; Francesconi, L. C.; Cutler, C. ²²⁷Th Labeled 3,4,3-(LI-1,2-HOPO): Optimized Synthesis, Biological Distribution and Dosimetry. J. Nucl. Med. 2021, 62, 1482.
(47) Ramdahl, T.: Bonge-Hansen, H. T.; Ryan, O. B.; Larsen, Å.; Herstad, G.; Sandberg, M.; Bjerke, R. M.; Grant, D.; Brevik, E. M.; Cuthbertson, A. S. An Efficient Chelator for
(48) Complexation of Thorium-227. Bioorganic Med. Chem. Lett. 2016, 26, 4318-4321. Deblonde, G. J. P.; Lohrey, T. D.; Booth, C. H.; Carter, K. P.; Parker, B. F.; Larsen, Å.; Smeets, R.; Ryan, O. B.; Cuthbertson, A. S.; Abergel, R. J. Solution Thermodynamics and Kinetics of Metal Complexation with a Hydroxypyridinone Chelator Designed for Thorium-227 Targeted Alpha Therapy. Inorg. Chem. 2018, 57, 14337-14346.
(49) Hagemann, U. B.; Ellingsen, C.; Schuhmacher, J.; Kristian, A.; Mobergslien, A.; Cruciani, V.; Wickstroem, K.; Schatz, C. A.; Kneip, C.; Golfier, S.; Smeets, R.; Uran, S.; Hennekes, H.; Karlsson, J.; Bjerke, R. M.; Ryan, O. B.; Mumberg, D.; Ziegelbauer, K.; Cuthbertson, A. S. Mesothelin-Targeted Thorium-227 Conjugate (MSLN-TTC): Preclinical Evaluation of a New Targeted Alpha Therapy for Mesothelin-Positive Cancers. Clin. Cancer Res. 2019, 25, 4723-4734.
(50) Hammer, S.; Hagemann, U. B.; Zitzmann-Kolbe, S.; Larsen, A.; Ellingsen, C.; Geraudie, S.; Grant, D.; Indrevoll, B.; Smeets, R.; Von Ahsen, O.; Kristian, A.; Lejeune, P.; Hennekes, H.; Karlsson, J.; Bjerke, R. M.; Ryan, O. B.; Cuthbertson, A. S.; Mumberg, D. Preclinical Efficacy of a PSMA-Targeted Thorium-227 Conjugate (PSMA-TTC), a Targeted Alpha Therapy for Prostate Cancer. Clin. Cancer Res. 2020, 26, 1985-1996.
(51) Lejeune, P.; Cruciani, V.; Berg-Larsen, A.; Schlicker, A.; Mobergslien, A.; Bartnitzky, L.; Berndt, S.; Zitzmann-Kolbe, S.; Kamfenkel, C.; Stargard, S.; Hammer, S.; Jørgensen, J. S.; Jackerott, M.; Nielsen, C. H.; Schatz, C. A.; Hennekes, H.; Karlsson, J.; Cuthbertson, A. S.; Mumberg, D.; Hagemann, U. B. Immunostimulatory Effects of Targeted Thorium-227 Conjugates as Single Agent and in Combination with Anti-PD-L1 Therapy. J. Immunother. Cancer 2021, 9, e002387.
(52) Lindén, O.; Bates, A. T.; Cunningham, D.; Hindorf, C.; Larsson, E.; Cleton, A.; Pinkert, J.; Huang, F.; Bladt, F.; Hennekes, H.; Oedegaardstuen, L. I.; Sturm, I.; McNamara. C. ²²⁷Th-Labeled Anti-CD22 Antibody (BAY 1862864) in Relapsed/Refractory CD22-Positive Non-Hodgkin Lymphoma: A First-in-Human, Phase I Study. Cancer Biother. Radiopharm. 2021, 36, 672-681.
(53) Roy, J.; Jagoda, E. M.; Basuli, F.; Vasalatiy, O.; Phelps, T. E.; Wong, K.; Ton, A. T.; Hagemann, U. B.; Cuthbertson, A. S.; Cole, P. E.; Hassan, R.; Choyke, P. L.; Lin, F. I. In Vitro and in Vivo Comparison of 3,2-HOPO Versus Deferoxamine-Based Chelation of Zirconium-89 to the Antimesothelin Antibody Anetumab. Cancer Biother. Radiopharm. 2021, 36, 316-325.
(54) Larsson, E.; Brolin, G.; Cleton, A.; Ohlsson, T.; Lindén, O.; Hindorf, C. Feasibility of Thorium-227/Radium-223 Gamma-Camera Imaging during Radionuclide Therapy. Cancer Biother. Radiopharm. 2020, 35, 540-548.
(55) Drude, N.; Tienken, L.; Mottaghy, F. M. Theranostic and Nanotheranostic Probes in Nuclear Medicine. Methods 2017, 130, 14-22.
(56) Schottelius, M.; Simeček, J.; Hoffmann, F.; Willibald, M.; Schwaiger, M.; Wester, H.-J. Twins in Spirit-Episode I: Comparative Preclinical Evaluation of [⁶⁸Ga]DOTATATE and [⁶⁸Ga]HA-DOTATATE. EJNMMI Res. 2015, 5, 22.
(57) Deri. M. A.; Zeglis, B. M.; Francesconi, L. C.; Lewis, J. S. PET Imaging with 99Zr: From Radiochemistry to the Clinic. Nucl. Med. Biol. 2013, 40, 3-14.
(58) Broer, L. N.; Knapen, D. G.; Suurs, F. V.; Moen, I.; Giesen, D.; Waaijer, S. J. H.; Indrevoll, B.; Ellingsen, C.; Kristian, A.; Cuthbertson, A. S.; de Groot, D.-J. A.; Cole, P. E.; de Vries, E. G. E.; Hagemann, U. B.; Lub-de Hooge, M. N. ⁸⁹Zr-3,2-HOPO-Mesothelin Antibody PET Imaging Reflects Tumor Uptake of Mesothelin Targeted ²²⁷Th-Conjugate Therapy in Mice. J. Nucl. Med. 2022, 63, jnumed. 121.263079.
(59) Southcott, L.; Li. L.; Patrick, B. O.; Stephan, H.; Jaraquemada-Peláez, M. D. G.; Orvig. C. [^{Nat/89}Zr][Zr(Pypa)]: Thermodynamically Stable and Kinetically Inert Binary Nonadentate Complex for Radiopharmaceutical Applications. Inorg. Chem. 2021, 60, 18082-18093.
(60) Feiner, I. V. J.: Brandt, M.; Cowell, J.; Demuth, T.; Vugts, D.; Gasser, G.; Mindt, T. L. The Race for Hydroxamate-Based Zirconium-89 Chelators. Cancers 2021, 13, 4466.
(61) Salih, A. K.; Raheem, S. J.; Garcia, M. D.; Ahiahonu, W. K.: Price, E. W. Design, Synthesis, and Evaluation of DFO-Em: A Modular Chelator with Octadentate Chelation for Optimal Zirconium-89 Radiochemistry. Inorg. Chem. 2022, 61, 20964-20976.
(62) Arnedo-Sanchez, L.; Smith, K. F.; Deblonde, G. J. P.; Carter, K. P.; Moreau, L. M.; Rees, J. A.; Tratnjek, T.; Booth, C. H.; Abergel, R. J. Combining the Best of Two Chelating Titans: A Hydroxypyridinone-Decorated Macrocyclic Ligand for Efficient and Concomitant Complexation and Sensitized Luminescence of f-Elements. Chempluschem 2021, 86, 483-491.
(63) Sturzbecher-Hoehne, M.; Choi, T. A.; Abergel, R. J. Hydroxypyridinonate Complex Stability of Group (IV) Metals and Tetravalent f-Block Elements: The Key to the Next Generation of Chelating Agents for Radiopharmaceuticals. Inorg. Chem. 2015, 54, 3462-3468.
(64) Deblonde, G. J. P.; Sturzbecher-Hoehne, M.; Rupert, P. B.; An, D. D.; Illy, M. C.; Ralston, C. Y.; Brabec, J.: De Jong, W. A.; Strong, R. K.; Abergel, R. J. Chelation and Stabilization of Berkelium in Oxidation State+IV. Nat. Chem. 2017, 9, 843-849.
(65) Sturzbecher-Hoehne, M.; Deblonde, G. J. P.; Abergel, R. J. Solution Thermodynamic Evaluation of Hydroxypyridinonate Chelators 3,4,3-LI(1,2-HOPO) and 5-LIO(Me-3,2-HOPO) for UO₂(VI) and Th(IV) Decorporation. Radiochim. Acta 2013, 101, 359-366.
(66) Deblonde, G. J. P.; Sturzbecher-Hoehne, M.; Abergel, R. J. Solution Thermodynamic Stability of Complexes Formed with the Octadentate Hydroxypyridinonate Ligand 3,4,3-LI(1,2-HOPO): A Critical Feature for Efficient Chelation of Lanthanide (IV) and Actinide (IV) Ions. Inorg. Chem. 2013, 52, 8805-8811.
(67) Deblonde, G. J. P.; Lohrey, T. D.; Abergel, R. J. Inducing Selectivity and Chirality in Group IV Metal Coordination with High-Denticity Hydroxypyridinones. Dalt. Trans. 2019, 48, 8238-8247.
(68) Xu, J.; Whisenhunt, D. W.; Veeck, A. C.; Uhlir, L. C.; Raymond, K. N. Thorium(IV) Complexes of Bidentate Hydroxypyridinonates. Inorg. Chem. 2003, 42, 2665-2674.
(69) Whisenhunt, D. W.; Neu, M. P.; Hou, Z.; Xu, J.; Hoffman, D. C.; Raymond, K. N. Specific Sequestering Agents for the Actinides. 29. Stability of the Thorium (IV) Complexes of Desferrioxamine B (DFO) and Three Octadentate Catecholate or Hydroxypyridinonate DFO Derivatives: DFOMTA, DFOCAMC, and DFO-1,2-HOPO. Comparative Stability of The. Inorg. Chem. 1996, 35, 4128-4136.
(70) Stasiuk, G. J.; Long, N. J. The Ubiquitous DOTA and Its Derivatives: The Impact of 1,4,7,10-Tetraazacyclododecane-1,4,7,10-Tetraacetic Acid on Biomedical Imaging. Chem. Commun. 2013, 49, 2732.
(71) Jackson, J. A.; Hungnes, I. N.; Ma, M. T.; Rivas, C. Bioconjugates of Chelators with Peptides and Proteins in Nuclear Medicine: Historical Importance, Current Innovations, and Future Challenges. Bioconjug. Chem. 2020, 31, 483-491.
(72) De León-Rodriguez, L. M.; Kovacs, Z. The Synthesis and Chelation Chemistry of DOTA-Peptide Conjugates. Bioconjug. Chem. 2008, 19, 391-402.
(73) Shieh, W. C.; Chen, Z.; Xue, S.; McKenna, J.; Wang, R. M.; Prasad, K.; Repič, O. Synthesis of Sterically-Hindered Peptidomimetics Using 4-(4,6-Dimethoxy-1,3,5-Triazine-2-Yl)-4-Methyl-Morpholinium Chloride. Tetrahedron Lett. 2008, 49, 5359-5362.
(74) Workman, D. G.; Hunter, M.; Dover, L. G.; Tétard, D. Synthesis of Novel Iron (III) Chelators Based on Triaza Macrocycle Backbone and 1-Hydroxy-2 (H)-Pyridin-2-One Coordinating Groups and Their Evaluation as Antimicrobial Agents. J. Inorg. Biochem. 2016, 160, 49-58.
(75) Workman, D. G.; Hunter, M.; Wang, S.; Brandel, J.; Hubscher, V.; Dover, L. G.; Tétard, D. The Influence of Linkages between 1-Hydroxy-2 (1H)-Pyridinone Coordinating Groups and a Tris(2-Aminoethyl)Amine Core in a Novel Series of Synthetic Hexadentate Iron (III) Chelators on Antimicrobial Activity. Bioorg. Chem. 2020, 95, 103465.
(76) Mahoney, S. J.; Johansen, M. B.; Moran, M. D.; Chassé, M.; Simms, R. W.; Burak, E.; Valliant, J. F. Macrocyclic Chelates and Uses Thereof. 2021, US2021/012697.
(77) Wilson, J. J.; Birnbaum, E. R.; Batista, E. R.; Martin, R. L.; John. K. D. Synthesis and Characterization of Nitrogen-Rich Macrocyclic Ligands and an Investigation of Their Coordination Chemistry with Lanthanum (III). Inorg. Chem. 2015, 54, 97-109.
(78) Natrajan, L. S.; Khoabane, N. M.; Dadds, B. L.; Muryn, C. A.; Pritchard, R. G.; Heath, S. L.; Kenwright, A. M.; Kuprov, I.; Faulkner, S. Probing the Structure, Conformation, and Stereochemical Exchange in a Family of Lanthanide Complexes Derived from Tetrapyridyl-Appended Cyclen. Inorg. Chem 2010, 49, 7700-7709.
(79) Abergel, R. J.; D'Aléo, A.; Ng Pak Leung, C.; Shuh, D. K.; Raymond, K. N. Using the Antenna Effect as a Spectroscopic Tool: Photophysics and Solution Thermodynamics of the Model Luminescent Hydroxypyridonate Complex [Eu^{III}(3,4,3-LI(1,2-HOPO))]⁻. Inorg. Chem. 2009, 48, 10868-10870.
(80) Desreux, J. F.; Merciny, E.; Loncin, M. F. Nuclear Magnetic Resonance and Potentiometric Studies of the Protonation Scheme of Two Tetraaza Tetraacetic Macrocycles. Inorg. Chem. 1981, 20, 987-991.
(81) Ascenso, J. R.; Delgado, R.; Fraústo da Silva, J. J. R. Nuclear Magnetic Resonance Studies of the Protonation Sequence of Cyclic Tetra-Azatetra-Acetic Acids. J. Chem. Soc., Perkin Trans. 2 1985, No. 6, 781-788.
(82) Burai, L.; Fábián, I.; Király, R.; Szilágyi, E.; Brücher, E. Equilibrium and Kinetic Studies on the Formation of the Lanthanide (III) Complexes, [Ce(Dota)]⁻ and [Yb(Dota)]⁻ (H₄Dota=1,4,7,10-Tetraazacyclododecane-1,4,7,10-Tetraacetic Acid). J. Chem. Soc. Dalt. Trans. 1998, No. 2, 243-248.
(83) Szigethy, G.; Raymond, K. N. The Influence of Linker Geometry in Bis(3-Hydroxy-N-Methyl-Pyridin-2-One) Ligands on Solution Phase Uranyl Affinity. Chem.—A Eur. J. 2011, 17.1818-1827.
(84) Szigethy, G.; Raymond, K. N. Hexadentate Terephthalamide (Bis-Hydroxypyridinone) Ligands for Uranyl Chelation: Structural and Thermodynamic Consequences of Ligand Variation (1). J. Am. Chem. Soc. 2011, 133, 7942-7956.
(85) Harris, W. R.; Carrano, C. J.; Raymond, K. N. Coordination Chemistry of Microbial Iron Transport Compounds. 16. Isolation, Characterization, and Formation Constants of Ferric Aerobactin. J. Am. Chem. Soc. 1979, 101, 2722-2727.
(86) Deblonde, G. J. P.; Sturzbecher-Hoehne, M.; Mason, A. B.; Abergel, R. J. Receptor Recognition of Transferrin Bound to Lanthanides and Actinides: A Discriminating Step in Cellular Acquisition of f-Block Metals. Metallomics 2013, 5, 619-626.
(87) Zhong, W.; Parkinson, J. A.; Guo, M.; Sadler, P. J. Unusual Features for Zirconium (IV) Binding to Human Serum Transferrin. JBIC J. Biol. Inorg. Chem. 2002, 7, 589-599.
(88) Choppin, G. R.; Erten, H. N.; Xia, Y.-X. Variation of Stability Constants of Thorium Citrate Complexes with Ionic Strength. Radiochim. Acta 1996, 74. 123-128.
(89) Krauskopf, K. B. Thorium and Rare-Earth Metals as Analogs for Actinide Elements. Chem. Geol. 1986, 55, 323-335.
(90) Tewari, R. C.; Srivastava, M. N. Potentiometric Determination of Stepwise Stability Constants of Zirconium, Thorium and Uranium Chelates of Asparagine and Glutamine. Talanta 1973, 20, 360-361.
(91) Kodama, M.; Koike, T.; Mahatma, A. B.; Kimura, E. Thermodynamic and Kinetic Studies of Lanthanide Complexes of 1,4,7,10,13-Pentaazacyclopentadecane-N,N′,N″,N′″,N″″-Pentaacetic Acid and 1,4,7,10,13,16-Hexaazacyclooctadecane-N,N′,N″,N″,N′″,N″″,N′″″-Hexaacetic Acid. Inorg. Chem. 1991, 30, 1270-1273.
(92) Savvin, S. B. Analytical Use of Arsenazo III. Determination of Thorium, Zirconium, Uranium and Rare Earth Elements. Talanta 1961. 8. 673-685.
(93) Woods, J. J.; Unnerstall, R.; Hasson, A.; Abou, D. S.; Radchenko, V.; Thorek, D. L. J.; Wilson. J. J. Stable Chelation of the Uranyl Ion by Acyclic Hexadentate Ligands: Potential Applications for ²³⁰U Targeted α-Therapy. Inorg. Chem. 2022, 61, 3337-3350.
(94) Hasson, A.; Jiang, W.; Benabdallah, N.; Lu, P.; Longtine, M. S.; Beattie, B. J.; Summer, L.; Zhang, H.; Wahl, R. L.; Abou, D. S.; Thorek, D. L. J. Radiochemical Quality Control Methods for Radium-223 and Thorium-227 Radiotherapies. Cancer Biother. Radiopharm. 2023. 38, 15-25.
(95) Borrebaek, J.; Krogh, C.; Larsen, R. H.; Melhus, K. B.; Dahle, J.; Bruland, Ø. S.; Valan, M. H. Preparation of Th²²⁷-Labeled Radioimmunoconjugates, Assessment of Serum Stability and Antigen Binding Ability. Cancer Biother. Radiopharm. 2007, 22, 431-437.
(96) Ivanov, P. I.; Collins, S. M.; van Es, E. M.; Garcia-Miranda, M.; Jerome, S. M.; Russell, B. C. Evaluation of the Separation and Purification of ²²⁷Th from Its Decay Progeny by Anion Exchange and Extraction Chromatography. Appl. Radiat. Isot. 2017, 124, 100-105.
(97) Collins. S. M.; Keightley, J. D.; Ivanov, P.; Arinc, A.; Jerome, S. M.; Fenwick, A. J.; Pearce, A. K. The Potential Radio-Immunotherapeutic α-Emitter ²²⁷Th-Part I: Standardisation via Primary Liquid Scintillation Techniques and Decay Progeny Ingrowth Measurements. Appl. Radiat. Isot. 2019, 145, 240-250.
(98) Vaughn, B. A.; Ahn, S. H.; Aluicio-Sarduy, E.; Devaraj, J.; Olson, A. P.; Engle, J.; Boros, E. Chelation with a Twist: A Bifunctional Chelator to Enable Room Temperature Radiolabeling and Targeted PET Imaging with Scandium-44. Chem. Sci. 2020, 11, 333-342.
(99) Aluicio-Sarduy, E.; Hernandez, R.; Olson, A. P.; Barnhart, T. E.; Cai, W.; Ellison, P. A.; Engle, J. W. Production and in Vivo PET/CT Imaging of the Theranostic Pair ^{132/135}La. Sci. Rep. 2019. 9, 10658.
(100) Mcinnes, L. E.; Rudd, S. E.; Donnelly, P. S. Copper, Gallium and Zirconium Positron Emission Tomography Imaging Agents: The Importance of Metal Ion Speciation. Coord. Chem. Rev. 2017, 352, 499-516.
(101) Bhatt, N. B.; Pandya, D. N.; Xu, J.; Tatum, D.; Magda, D.; Wadas, T. J. Evaluation of Macrocyclic Hydroxyisophthalamide Ligands as Chelators for Zirconium-89. PLOS One 2017, 12, e0178767.
(102) Pandya, D. N.; Pailloux, S.; Tatum, D.; Magda, D.; Wadas, T. J. Di-Macrocyclic Terephthalamide Ligands as Chelators for the PET Radionuclide Zirconium-89. Chem. Commun. 2015. 51, 2301-2303.
(103) Bhatt, N.; Pandya, D.; Wadas, T. Recent Advances in Zirconium-89 Chelator Development. Molecules 2018, 23, 638.
(104) Holland, J. P. Predicting the Thermodynamic Stability of Zirconium Radiotracers. Inorg. Chem. 2020, 59, 2070-2082.
(105) N. Tinianow, J.; Pandya, D. N.; Pailloux, S. L.; Ogasawara, A.; Vanderbilt, A. N.; Gill, H. S.; Williams, S.-P.; Wadas, T. J.; Magda, D.; Marik, J. Evaluation of a 3-Hydroxypyridin-2-One (2,3-HOPO) Based Macrocyclic Chelator for ⁵⁹Zr⁴⁺ and Its Use for ImmunoPET Imaging of HER2 Positive Model of Ovarian Carcinoma in Mice. Theranostics 2016, 6, 511-521.

### Example 1

### Macrocyclic 1,2-Hydroxypyridinone-based Chelators as Potential Ligands for Thorium-227 and Zirconium-89 Radiopharmaceuticals

Thorium-227 (²²⁷Th) is an α-emitting radionuclide that has shown preclinical and clinical promise for use in targeted α-therapy (TAT), a type of molecular radiopharmaceutical treatment that harnesses high energy α particles to eradicate cancerous lesions. Despite these initial successes, there still exists a need for bifunctional chelators that can stably bind thorium in vivo. Towards this goal, we have prepared two macrocyclic chelators bearing 1,2-hydroxypyridinone chelating groups. Both chelators can be synthesized in less than 6 steps from readily available starting materials, which is an advantage over currently available platforms. The complex formation constants (log βmlh) of these ligands with Zr4+ and Th4+, measured by spectrophotometric titrations, are greater than 34 for both chelators indicating the formation of exceedingly stable complexes. Radiolabeling studies were performed to show that these ligands can bind [²²⁷Th]Th4+ at concentrations as low as 10-6 M and serum stability experiments demonstrate the high kinetic stability of the formed complexes under biological conditions. Identical experiments with zirconium-89 (⁸⁹Zr), a positron-emitting radioisotope used for positron emission tomography (PET) imaging, demonstrate that these chelators can also effectively bind Zr4+ with high thermodynamic and kinetic stability. Collectively, the data reported herein highlight the suitability of these ligands for use in ⁸⁹Zr/²²⁷Th paired radioimmunotheranostics.

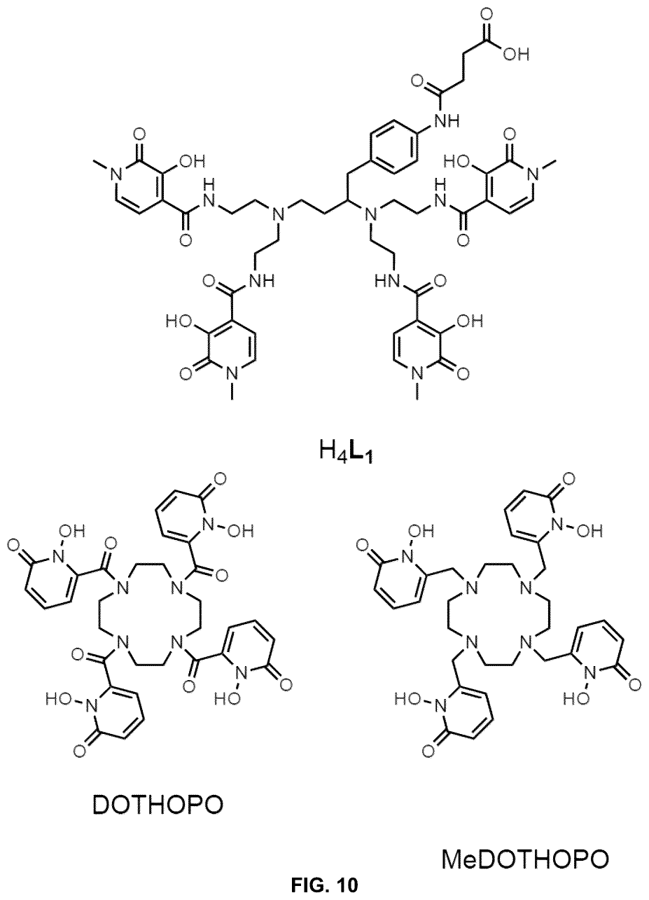

In this report, we evaluate the suitability of two macrocyclic octadentate chelators, 1,4,7,10-tetraazacyclododecane-1,4,7,10-tetra(carbonyl)tetrakis(1-hydroxy-2-pyridinone) (DOTHOPO) 62 and 1,4,7,10-tetraazacyclododecane-1,4,7,10-tetra(methylene)tetrakis(1-hydroxy-2-pyridinone) (MeDOTHOPO), for chelation of ²²⁷Th and ⁸⁹Zr (FIG. 10). These ligands were chosen based on the high affinity of the 1,2-HOPO moiety for tetravalent metals⁶³⁻⁶⁹ and their overall synthetic accessibility compared to the current state-of-the-art chelator L1. Solution thermodynamics and radiolabeling studies found that these ligands possess high affinity for +4 metals and rapidly complex [²²⁷Th]Th⁴⁺ under mild conditions at concentrations as low as 10⁻⁶ M. Serum stability experiments confirm that the resulting complexes possess excellent in vitro stability. Parallel experiments were also performed with [⁸⁹Zr]Zr⁴⁺ to demonstrate that these ligands are also effective for in vivo chelation of this radionuclide. The results presented herein describe promising chelators for the future development of ⁸⁹Zr and ²²⁷Th radiopharmaceuticals, especially when considering the relatively-well established synthetic methods to append a bioconjugation site on the tetraazacyclododecane macrocyclic scaffold.⁷⁰⁻⁷²

### Results and Discussion

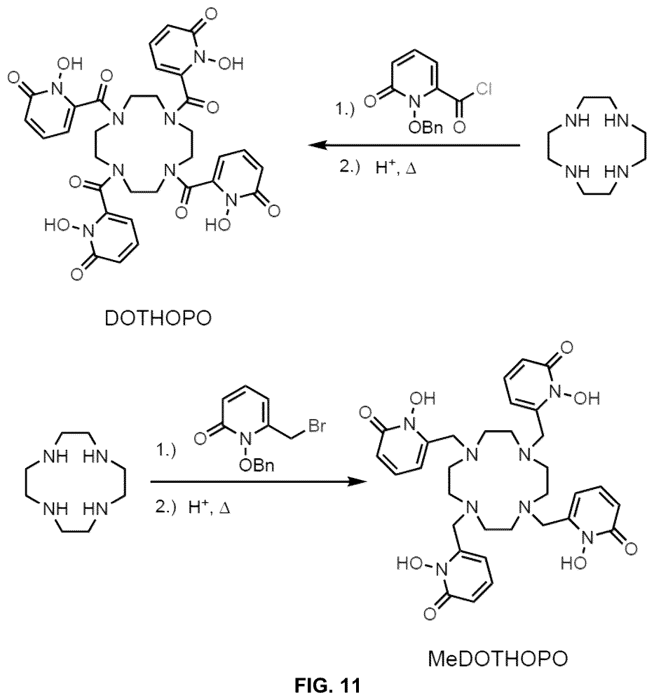

We have previously reported that the synthesis of DOTHOPO via the reaction between benzyl-protected 1,2-HOPO acid chloride and cyclen in CH₂Cl₂ produces an undesired side product containing only three 1,2-HOPO groups.₆₂ This impurity was also observed when the reaction was performed in refluxing THE or when the acyl fluoride 1,2-HOPO derivative was used in place of the acyl chloride species. Performing the reaction in the presence of the amide coupling reagent 4-(4,6-dimethoxy-1,3,5-triazine-2-yl)-4-methyl-morpholinium chloride (DMTMM), which performs well when coupling sterically hindered amines, 73 also produced a mixture of the two compounds. Our strategy to selectively isolate the tetrasubstituted compound took advantage of the differing aqueous solubility of the two compounds; successive fractional crystallization of the crude product from ethanol/water mixtures gave analytically pure tetrasubstituted macrocycle in approximately 70% yield based on cyclen. Deprotection under acidic conditions yielded DOTHOPO in two steps from known starting materials (FIG. 11).

It has been shown that the tertiary amide groups of macrocycles bearing 1,2-HOPO groups have a significant impact on the metal-binding ability of the resulting chelators. 74,75 To investigate the role of this functionality in the coordination of tetravalent metals we synthesized the ligand MeDOTHOPO, where the tertiary amide groups are replaced by tertiary amines. This chelator was prepared in high yield via N-alkylation of cyclen using an alkyl bromide derivative of 1,2-HOPO (S8), which was synthesized in four steps from the known 1,2-HOPO carboxylic acid starting material (S1). Subsequent deprotection of the benzyl groups was performed under acidic conditions (FIG. 11). It should be noted that the preparation of MeDOTHOPO has been recently described in a patent,₇₆ but a thorough investigation of the coordination chemistry of this ligand has not been reported. The synthesis of both chelators is significantly simpler than that of L₁, which requires 17 steps.⁴⁷

Both chelators were characterized by standard analytical techniques including mass spectrometry, elemental analysis, ultra-performance liquid chromatography (UPLC), and NMR spectroscopy. The 1H NMR of DOTHOPO in D2O at pD~5 showed the presence of multiple conformers in solution. The exchange between conformations is slow on the NMR time scale, as indicated by the broadened peaks corresponding to the 1,2-HOPO units. The presence of multiple HOPO environments appears to be the result of extensive hydrogen bonding; addition of NaOD to pD~10 yielded much sharper and well-defined peaks with one major 1,2-HOPO environment present in solution. The signals corresponding to the cyclen backbone are poorly resolved under both conditions, suggesting that the cyclen ring is highly flexible. The 1H NMR spectrum of MeDOTHOPO at pD 5 displays well resolved signals, which suggests that interconversion between conformations is fast in solution.

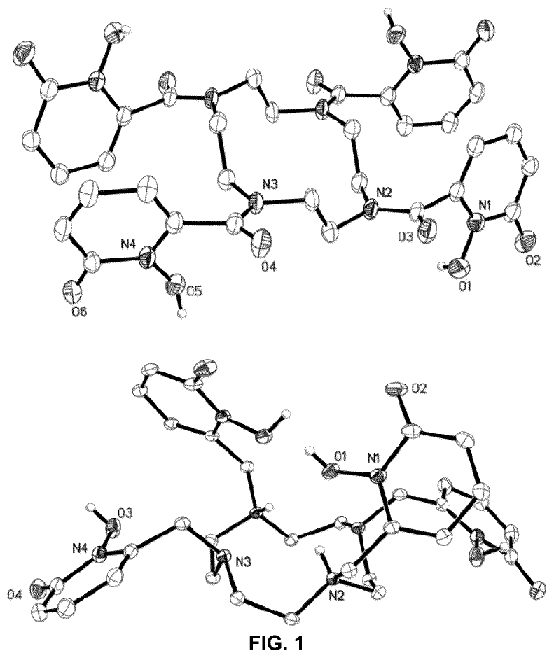

The solid-state structures of both chelators were determined by X-ray diffraction (FIG. 1). Crystals of DOTHOPO were obtained by slow evaporation of an aqueous solution of the compound. Under these conditions, the ligand crystallizes in the space group P-1. Similar to other DOTA derivatives,^{77,78} the ligand adopts an open chair-like conformation. The center of the 12-membered macrocycle lies on a crystallographic inversion center such that one half of the ligand is present in the asymmetric unit. The tertiary amide groups are not coplanar with their respective 1,2-HOPO units (torsion angles ranging from 69.1°-106.1°), indicating that the π systems of the amide functional groups and the 1,2-HOPO donors are not conjugated. This observation is likely due to the steric constraints imposed by the presence of the tertiary amide units.₆₂ Crystallization of MeDOTHOPO as the HBr salt was performed by slow evaporation of a solution of the chelator from 1:1 EtOH/H₂O in the presence of HBr. The ligand crystallizes such that the center of the macrocycle lies on a crystallographic two-fold axis. In contrast to DOTHOPO, the macrocyclic core of MeDOTHOPO adopts a puckered conformation, which facilitates intramolecular hydrogen bonding interactions between the protonated amines and two of the 1,2-HOPO groups. Analysis of the C═O_{HOPO} and N—O_{HOPO} interatomic distances in the structures revealed no significant differences between the two chelators.

The protonation constants (pKa) of the synthesized ligands were determined potentiometrically in 0.1 M KCl. Titration of DOTHOPO over the pH range of 2.5-11 revealed four protonation equilibria corresponding to sequential deprotonation of each of the four 1,2-HOPO units (Table 1). The obtained protonation constants of DOTHOPO are similar to those of the linear spermine-backbone analogue 3,4,3-LI-(1,2-HOPO),⁷⁹ indicating that the macrocyclic backbone does not greatly affect the acidity of the 1,2-HOPO units. Titration of MeDOTHOPO over the same pH range revealed a total of six protonation equilibria with four of the constants tentatively assigned to protonation of the HOPO donor arms. Based on previous studies involving ligands bearing the tetraazacyclododecane backbone,⁸⁰⁻⁸² the remaining two pKa values likely correspond to protonation of two of the amine groups present in the macrocycle (Table 1). The 1,2-HOPO donors of MeDOTHOPO are approximately 1 pKa unit more basic than those of 3,4,3-LI-(1,2-HOPO) or DOTHOPO. The overall acidity of a chelator can be characterized by the sum of the pKa values (ΣpKa) corresponding to the protonation of the chelating groups; a lower value of ΣpKa indicates a more acidic ligand.^{83,84} Comparison of the ΣpKₐ values between these three ligands highlights the decreased acidity of MeDOTHOPO (ΣpKₐ=24.96) compared to L₁ (ΣpKₐ=21.99),₄₈ and DOTHOPO (ΣpKₐ=21.34).

TABLE 1
Protonation constants of L1, DOTHOPO, and MeDOTHOPO.^{a}

| | L₁^{b} | DOTHOPO | MeDOTHOPO |
|---|---|---|---|
| pKₐ₁ | 2.93(35) | 6.43(9) | 10.52(6) |
| pKₐ₂ | 4.39(8)^{c} | 5.46(1) | 9.1(2) |
| pKₐ₃ | 4.73(34) | 5.05(3) | 7.79(8) |
| pKₐ₄ | 5.97(14) | 4.28(6) | 6.63(3) |
| pKₐ₅ | 6.90(5) | — | 6.06(3) |
| pKₐ₆ | 7.74(6) | — | 4.41(6) |
| pKₐ₇ | 9.58(16) | — | — |
| ΣpKₐ^{d} | 21.99 | 21.34 | 24.96 |

^{a}25° C., I = 0.1M KCl. The number given in parentheses corresponds to the standard deviation of the last digit of the stability constant (n = 5).
^{b}Data taken from Ref. ⁴⁸
^{c}Bold values correspond to pKa values tentatively assigned to protonation of the HOPO units.
^{d}ΣpKₐ, corresponds to the sum of the pKₐ values tentatively assigned to protonation of the HOPO units.

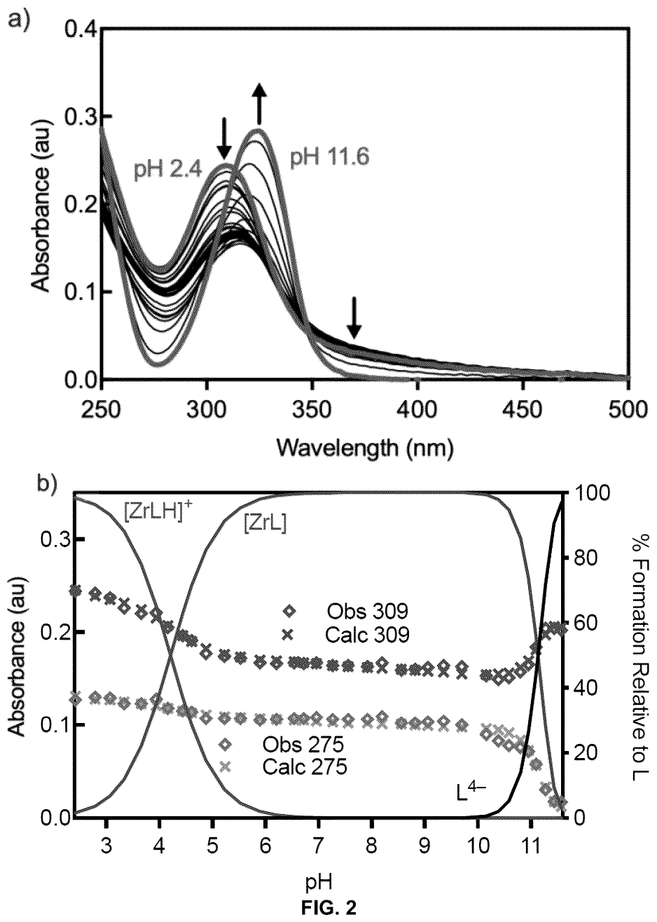

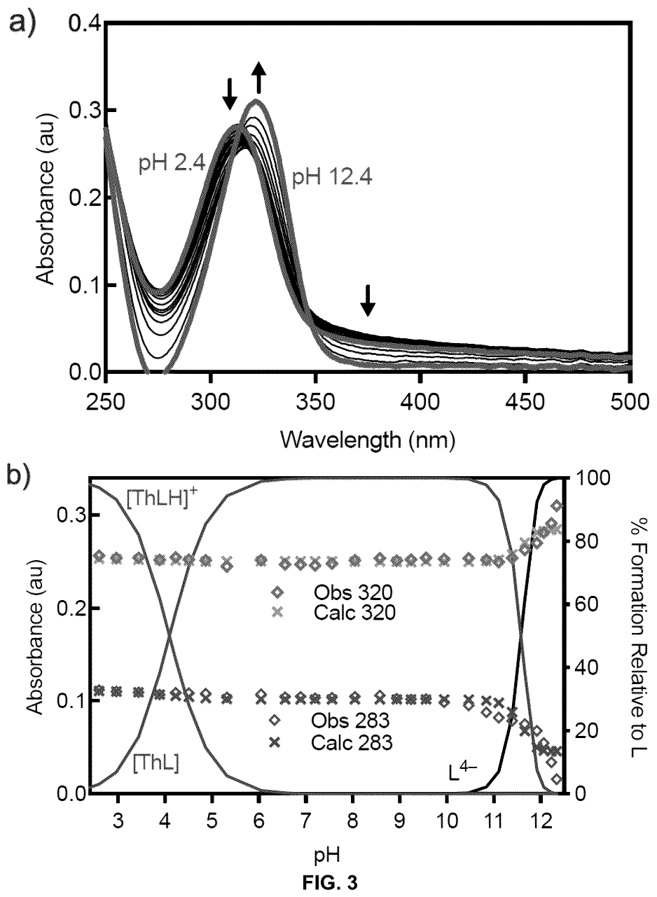

We next sought to evaluate the thermodynamic stabilities of the Zr⁴⁺ and Th⁴⁺ complexes of DOTHOPO and MeDOTHOPO by measuring their cumulative formation constants (log βₘₗₕ, see Supplementary Information for details). Initial experiments showed complex formation even in 1 M HCl, which is consistent with the extremely high affinity of the 1,2-HOPO group for tetravalent metals.^{63-66,69} Our successful evaluation of the thermodynamic stability constants of these ligands with the tetravalent metals of interest took advantage of the preference of these metals for the OH₋ ion at high pH. Above pH 10, the complexes dissociate to yield the free ligand and metal-hydroxide species, which could be leveraged to determine the stability constants of the Zr⁴⁺ and Th⁴⁺ complexes via UV-vis spectrophotometric titrations (FIGS. 2 and 3). The results of the titration experiments are displayed in Table 2 and FIGS. 2-3. The pH dependent species distribution diagrams for DOTHOPO with Zr⁴⁺ and Th⁴⁺ are provided in the Supporting Information. Importantly, the metal remains completely bound by the chelator below pH 10 and the major species present at pH 7.4 is the 1:1 ML complex for both metals. The higher affinity of DOTHOPO for the smaller Zr⁴⁺ over Th₄₊ is in agreement with other HOPO-based systems.^{48,63,66} The Zr₄₊ and Th₄₊ complexes of MeDOTHOPO did not dissociate appreciably up to pH 12 and competition titrations using DTPA were unsuccessful as this chelator could not compete with MeDOTHOPO for complexation of either metal in solution. As such, the log βₘₗₕ values of MeDOTHOPO with Zr⁴⁺ and Th⁴⁺ were predicted to be ≥51 and ≥53, respectively, using Hyss (Table 2).

The affinity of these ligands for Fe₃₊ was explored as a representative endogenous metal that could compete with Th⁴⁺ in vivo. The stability constants of DOTHOPO and MeDOTHOPO with Fe³⁺ were determined by incremental spectrophotometric titrations, similar to those described for Zr⁴⁺ and Th⁴⁺ in the Supporting Information. Refinement of the titration data revealed the presence of two species corresponding to [ML]⁻ and [MLH] complexes (Table 2).

TABLE 2
Summary of the cumulative formation constants of the Fe³⁺,
Zr⁴⁺, and Th⁴⁺ complexes of the investigated ligands.a

| | | | log βₘᵢₕ | |
|---|---|---|---|---|
| Metal | Species | L1^{b} | DOTHOPO | MeDOTHOPO |
| Fe³⁺ | [FeL]⁻ | 26.9(3) | 27.8(4) | 30.3(2) |
| | [FeLH] | 31.2(4) | 33.3(6) | 39.5(4) |
| Zr⁴⁺ | [ZrL] | 44.7(1) | 42.9(4) | ≥51^{c} |
| | [ZrLH]⁺ | 50.6(2) | 47.0(6) | — |
| Th⁴⁺ | [ThL] | — | 34.4(1) | ≥53^{c} |
| | [ThL]⁻ | 41.7(3) | — | — |
| | [ThLH]⁺ | — | 38.5(1) | — |
| | [ThLH] | 45.9(4) | — | — |
| | [ThH₋₁L]²⁻ | 32.4(4) | — | — |

a25° C., I = 0.1M KCl
^{b}Data for L₁ were taken from reference 48
^{c}Predicted. See text for details.

The log βₘₗₕ values reported above are pH-independent absolute stability constants. Because these ligands have differing pKa values, competition between H⁺ and the metal of interest under different pH conditions will give rise to different pH-dependent conditional stability constants that are not accurately described by log βML values. To mitigate this issue and allow for direct comparison between ligands, we calculated the pM values (pM=−log[M_{free}], where M_{free}=solvated metal ions free of complexation by ligand or hydroxide when [L]=10⁻⁵ M and [M]=10⁻⁶ M)⁸⁵ for each ligand with the Fe³⁺, Zr⁴⁺, and Th⁴⁺ (Table 3). Like L1, DOTHOPO and MeDOTHOPO form complexes with Zr⁴⁺ and Th⁴⁺ that are extremely stable compared to those formed by endogenous chelating molecules, 86-90 suggesting that they should not undergo transchelation in vivo. Additionally, the pM values of these chelators with Zr⁴⁺ and Th⁴⁺ are approximately 20 log units greater than those for Fe³⁺, suggesting that this transition metal should not be able to displace these tetravalent metals in the body, even at the largely higher concentrations encountered under physiological conditions (Table 3).

TABLE 3
Tabulated pM valuesa for L1, DOTHOPO, and MeDOTHOPO
with Fe³⁺, Zr⁴⁺, and Th⁴⁺ at pH
7.4 (25° C., I = 0.1M KCl).

| | Fe³⁺ | Zr⁴⁺ | Th⁴⁺ |
|---|---|---|---|
| L₁ | 25.1 | 42.9 | 39.9 |
| DOTHOPO | 28.7 | 43.7 | 35.3 |
| MeDOTHOPO | 27.6 | >46^{b} | >48^{b} |

apM = -log[M]_{free} ([M]ₜₒₜ = 10⁻⁶M; [L]ₜₒₜ = 10⁻⁵M; pH = 7.4; 25° C.; I = 0.1M KCl)
^{b}Predicted using log βₘₗₕ values reported in Table 2.

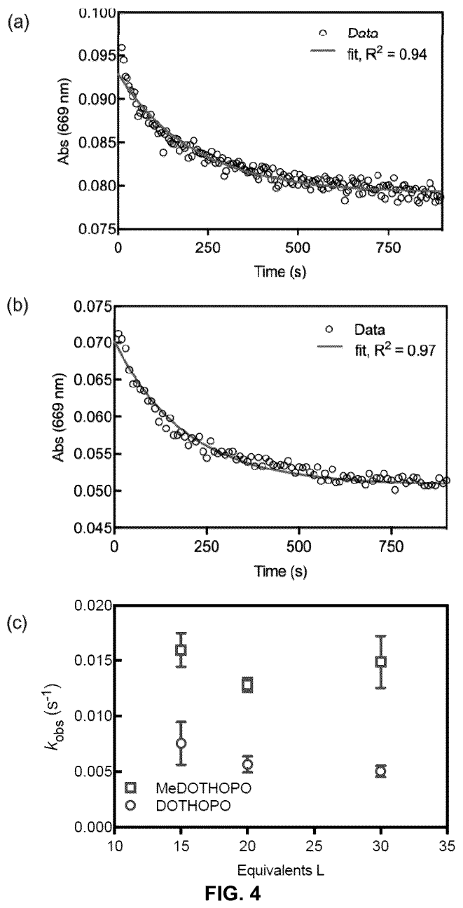

Another important aspect of chelators for radiopharmaceutical applications is the kinetics of complexation with the radiometal of interest. A ligand must be able to bind the radioactive metal ion rapidly and completely to minimize the extent of radioactive decay before administration in vivo. As noted above, DOTA displays slow formation rates with metal ions at room temperature and requires high radiolabeling temperatures (90° C.).⁹¹ These conditions are unsuitable for radiotherapeutic studies involving biological macromolecules, such as antibodies, which are temperature-sensitive. To access the suitability of DOTHOPO and MeDOTHOPO in this context, we sought to determine the kinetics of Th⁴⁺ complexation of these chelators in aqueous solution at 25° C. Initial experiments revealed only small spectral differences between the free ligands and their Th⁴⁺ complexes in the presence of excess ligand, making it difficult to accurately monitor the complexation reaction directly by UV-vis spectroscopy. Following literature precedent,³⁵ the relative rate of metal complexation by these ligands could be studied indirectly using the dye Arsenazo III, which forms highly colored complexes with Th⁴⁺ with a prominent absorbance band at 669 nm.⁹² The addition of excess chelator to solutions of Th⁴⁺ and Arsenazo III caused the dye to be displaced and the formation of the metal-chelator complex could be monitored by the disappearance of the absorbance features corresponding to the metal-dye compound (FIG. 4).

The aim of these experiments was to determine a second-order rate constant for the complexation reaction of Th⁴⁺ by DOTHOPO and MeDOTHOPO. This information can be obtained by performing the dye displacement reaction under pseudo-first order conditions using increasing concentrations of excess ligand.^{35,93} Unfortunately, we did not observe a clear rate dependence on the ligand concentration under conditions ranging from 15-30 equivalents metal (FIG. 4). Based on these results, we hypothesize that the rate-limiting step of the displacement reaction is likely the dissociation of the dye-metal complex rather than ligand association in the presence of rapidly complexing ligands like those reported here. For comparison purposes, the second-order rate constant of complexation was calculated using the reaction of 15 equiv. of ligand relative to metal (Table 4, see SI for details). The [ThL] (L=DOTHOPO, MeDOTHOPO) complexes form rapidly in aqueous solution. The second-order halflives, calculated for 10 μM each of metal and ligand, are 30 and 18 min for DOTHOPO and MeDOTHOPO respectively. The faster complexation kinetics of MeDOTHOPO compared to DOTHOPO can likely be attributed to the increased flexibility of the ligand. Both chelators performed significantly better than DTPA and DOTA, the latter of which did not show any complexation over 24 h at 25° C. Previous work has shown that under similar conditions, DOTA does not displace Arsenazo III from Th⁴⁺ at room temperature after 2 months.³⁵ Attempts to perform similar experiments with Zr⁴⁺ were thwarted by the dissociation of the Arsenazo III complex with Zr⁴⁺ under these conditions.

TABLE 4
Indirect kinetic dataa for Th⁴⁺ binding
by DOTHOPO, MeDOTHOPO, DTPA, and DOTA.

| Ligand | k₂ (M⁻¹ s⁻¹) | t_{1/2} (min) |
|---|---|---|
| DOTHOPO | 54.2 ± 2.8 | 30 |
| MeDOTHOPO | 89.2 ± 8.5 | 18 |
| DTPA | 7.9 ± 0.9 | 211 |
| DOTA | NR^{b} | — |

aConditions: 5 μM Th⁴⁺, 25 μM Arsenazo III. 75 μM ligand. 100 mM HEPES (pH 7.4), 25° C.
^{b}No reaction observed over 24 h at 25° C.

The high thermodynamic stability of the Zr⁴⁺ and Th⁴⁺ complexes with DOTHOPO and MeDOTHOPO motivated us to investigate ability of these chelators to be radiolabeled with [²²⁷Th]Th4+ and [89Zr]Zr4+ and the kinetic stability of the resulting complexes under biologically relevant conditions. These experiments required a robust and reliable method to purify ²²⁷Th from its parent isotope ²²⁷Ac and daughter products including ²²³Ra. Radiochemical purification of ²²⁷Th typically involves anion exchange chromatography techniques. In nitric acid solutions, thorium displays high affinity towards anion exchange resins, which can be attributed to the formation of [Th(NO₃)₆]²⁻. Actinium and daughters in the ²²⁷Th decay chain do not form anionic complexes under these conditions and can be removed by washing the column with HNO₃. The thorium is then eluted with dilute HCl. In our hands, attempts to purify ²²⁷Th using AG-1×8 anion exchange resin, the most common resin reported for this purification,^{16,33,47,94,95} resulted in a maximum recovery of 45% when eluting with 0.1 M HCl. Other experiments using HCl concentrations ranging from 0.05-6 M did not improve thorium recovery, making this resin unsuitable for purification of ²²⁷Th for medical applications. Our successful purification of ²²⁷Th involved the use of TEVA extraction chromatography resin in place of AG-1×8 anion exchange resin.^{96,97} Briefly, a 3 M HNO₃ solution containing ²²⁷Ac, ²²⁷Th, and their decay progeny was loaded onto a pre-packed cartridge of TEVA resin. The resin was then washed with 3 M HNO₃ to remove ²²⁷Ac, ²²³Ra, and daughters, which could be recycled or used for other studies. Subsequent elution with 0.1 M HCl yielded ²²⁷Th with >90% recovery, which is significantly improved over AG-1×8 resin. The radiochemical purity of the final product was >99% as determined by gamma spectroscopy (²²³Ra) and liquid scintillation counting (²²⁷Ac).

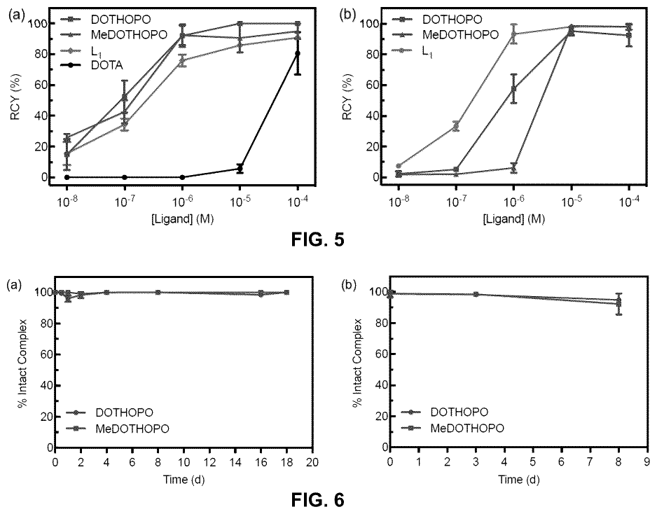

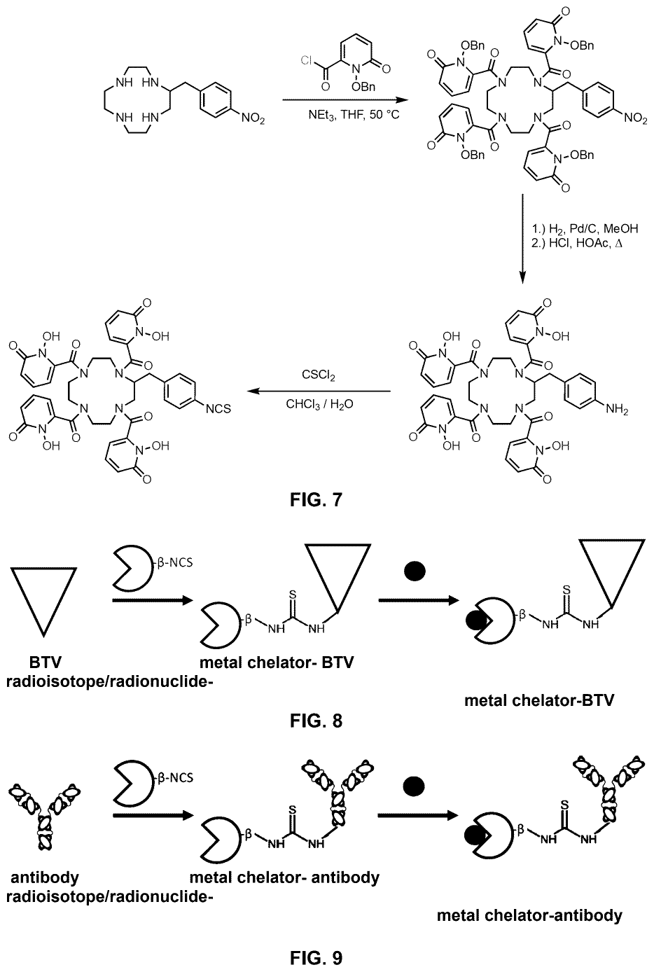

With a reliable and robust method to purify ²²⁷Th on hand, concentration-dependent radiolabeling studies were carried out to determine the radiolabeling ability of DOTHOPO and MeDOTHOPO in comparison to the two most established chelators, L1 and DOTA. Briefly, these experiments were performed by incubating 0.25 μCi of [²²⁷Th]Th⁴⁺ with varying concentrations of ligand in 0.25 M citrate buffer (pH 5) at 25° C. for 30 minutes. The radiochemical yield (RCY) was determined by instant thin layer chromatography (iTLC). The results are summarized in FIG. 5. Both chelators achieved >92% complexation at a ligand concentration of 10⁻⁶ M. The apparent molar activity (Am), calculated as twice the number of moles of chelator required to complex 50% of the radioactivity,^{98,99} was determined to be 13.8 mCi/μmol (509 MBq/μmol) and 11.6 mCi/μmol (428 MBq/μmol) for DOTHOPO and MeDOTHOPO respectively. Both ligands performed comparably to, if not slightly better than, L1 (Am=7.2 mCi/μmol; 266 MBq/μmol). In contrast, DOTA only showed partial incorporation of [²²⁷Th]Th⁴⁺ at the highest tested concentration of 10⁻⁴ M and with an Am of <0.07 mCi/μmol (<2.66 MBq/μmol), which agrees with previous studies.⁴⁰ Radiolabeling experiments with [⁸⁹Zr]Zr4+ (FIG. 5) demonstrated that DOTHOPO and MeDOTHOPO were quantitatively radiolabeled with this radioisotope at 10-5 M with diminishing efficiency at lower concentrations. DOTHOPO (Am=31.8 mCi/μmol; 1175.5 MBq/μmol) performed considerably better than MeDOTHOPO (Am=16.3 mCi/μmol; 602.9 MBq/μmol). This is likely due to the higher basicity of MeDOTHOPO, as it has been shown that chelators with higher protonation constants (pKa≥6) poorly radiolabel [⁸⁹Zr]Zr⁴⁺ due to metal hydrolysis at the relatively high pH conditions required for ligand deprotonation. 45,100 Of the three ligands examined, L1 demonstrated the highest specific activity with Am=158.3 mCi/μmol (5858.1 MBq/μmol). The acyclic nature, and thus greater flexibility, of L1 may contribute to its improved radiolabeling compared to DOTHOPO.¹⁰¹⁻¹⁰⁴

Having observed the excellent ability of these chelators to be radiolabeled by ⁸⁹Zr and ²²⁷Th, we next sought to investigate their stability in human serum to mimic conditions experienced by the radiopharmaceutical agent after administration in the body. Briefly, the radiolabeled complexes of DOTHOPO and MeDOTHOPO were incubated in human serum to mimic the conditions experienced by the radiopharmaceutical agent after administration in vivo. The amount of intact complex remaining in serum was determined by iTLC. In the case of ²²⁷Th, ingrowth of ²²³Ra and other α-emitting daughters in the ²²⁷Th decay chain made it difficult to definitively distinguish between complexed and free ²²⁷Th using the TLC scanner. To mitigate this challenge, the iTLC strips were developed as normal and then cut in half. The amount of ²²⁷Th at the top (free [²²⁷Th]Th⁴⁺) and bottom (complexed [²²⁷Th]Th⁴⁺) of the strip was determined by gamma spectroscopy. As depicted in FIG. 6, the [²²⁷Th]Th⁴⁺ complexes of both ligands remained >⁹⁹% intact and the [89Zr]Zr⁴⁺ complexes remained >92% intact over the period investigated, indicating that these chelators are exceptional candidates for further development of radiopharmaceuticals based on ⁸⁹Zr and ²²⁷Th. Chelators designed for ²²⁷Th generally lack same in vivo stability with ⁸⁹Zr,^{53,58,105} which further highlights the potential of these ligands as chelators for theranostic applications.

### CONCLUSIONS

The work presented herein establishes DOTHOPO and MeDOTHOPO as promising chelators for ²²⁷Th. Both ligands were synthesized in two (DOTHOPO) and six (MeDOTHOPO) steps, which is a significant improvement compared to other chelators designed for this purpose. Thorough characterization of these ligands revealed how removal of the tertiary amide group influences both the structure of the chelator in the solution and solid state and its relative acidity. The 1,2-HOPO donors of MeDOTHOPO are almost four orders of magnitude less acidic than those of DOTHOPO. Solution thermodynamic studies demonstrate that the compounds presented here possess extremely high affinity for tetravalent metals. The stability constants of MeDOTHOPO with Zr⁴⁺ and Th⁴⁺ are sufficiently high such that they could not be determined by direct titration or competition methods and are estimated to be >51 for both metals. These results demonstrate that removal of the tertiary amide groups significantly improves the thermodynamic stability of chelators bearing 1,2-HOPO groups with tetravalent metals.

Radiolabeling experiments with ²²⁷Th demonstrate that DOTHOPO and MeDOTHOPO can effectively bind this isotope at concentrations as low as 10⁻⁶ M within 30 min under mild conditions (room temperature, pH 5). These chelators can also be efficiently radiolabeled with ⁸⁹Zr, a positron-emitting radioisotope that holds potential as a diagnostic imaging isotope for use in conjunction with ²²⁷Th. Even though MeDOTHOPO possesses the largest pZr of the ligands investigated, it performed the worst in the ⁸⁹Zr radiolabeling experiments. This result highlights how thermodynamic stability alone cannot be used to predict the suitability of a chelator for radiotherapeutic applications. We further evaluated the kinetic stabilities of the [⁸⁹Zr]Zr⁴⁺ and [²²⁷Th]Th⁴⁺ complexes of the synthesized chelators in human serum. All the complexes remain sufficiently stable (>99% for ²²⁷Th and >92% for ⁸⁹Zr) in human serum. The work reported herein demonstrates how ligand design strategies can be leveraged to develop systems that can effectively bind [⁸⁹Zr]Zr⁴⁺ and [²²⁷Th]Th⁴⁺ to harness the theranostic potential of this pair of radioisotopes. Current work in our lab is focused on the synthesis, characterization, and biological evaluation of bifunctional analogues of these chelators to further study their applicability towards developing ²²⁷Th and ⁸⁹ Zr radiopharmaceuticals.

### Supporting Information:

All isotopes of thorium emit ionizing radiation. Actinium-227 (227Ac) and its daughters present serious health threats due to their α-, β-, and γ-radiation. Zirconium-89 is a positron-emitting isotope that produces high energy (909.15 keV, 99.04% intensity) γ-radiation in its decay. All materials were handled in a facility designed in accordance with appropriate shielding and safety protocols including HEPA-filtered fume hoods, lead bricks with a thickness of >2.5 cm, and radiation shields with leaded glass windows.

Unless otherwise indicated, all reagents were obtained commercially and used as received. All acids were of Optima grade (Fisher Scientific). All aqueous solutions and buffers were prepared with MilliQ water (18 MΩ·cm). Organic solvents were stored over activated 4 Å molecular sieves with no other attempts to exclude air or moisture.

NMR spectra were acquired at 25° C. using either a Bruker AVANCE NEO 300 MHz spectrometer equipped with a 5 mm BBO probe or a Bruker AVANCE NEO 500 MHz spectrometer equipped with a PA 5 mm BBO probe. The ¹H and ¹³C spectra were referenced to the residual solvent peak and chemical shifts are reported in ppm relative to Me4Si. The ¹⁹F spectra were referenced indirectly to the corresponding 1H spectrum using the absolute reference function as implemented in MestReNova (Mestrelab Research, S.L.) and are reported in ppm relative to CFCl₃. Spectra acquired in D2O were spiked with acetone as an internal reference. The splitting of proton resonances in the reported 1H NMR spectra is defined as s=singlet, d=doublet, t=triplet, m=multiplet, and br=broad. Elemental analysis was performed at the Microanalytical Facility in the College of Chemistry at UC Berkeley. Static UV-vis spectra of nonradioactive samples were recorded using a Varian Cary 5G UV-Vis-NIR spectrophotometer in 1 cm quartz cuvettes (Agilent Technologies, Inc) and spectra of radioactive samples were acquired in 1 cm UV-transparent cuvettes using a FLAME-T-XR1-ES spectrometer fitted with a CUV cuvette holder and a DH-2000 light source (Ocean Optics, Inc.) The spectrometer was controlled with Ocean View software (Ocean Optics, Inc.).

UPLC-MS were acquired using a UPLC Waters Xevo system interfaced with a QTOF mass spectrometer (Waters Corporation) in Micromass Z-spray geometry. UV-vis traces were monitored at 300 and 220 nm The UPLC system was fitted with an ACQUITY Premier HSS T3 Column with a VanGuard FIT column guard system (particle size: 1.8 μm; column size: 2.1×100 mm). Methods were performed using a flow rate of 0.5 mL min⁻¹ with a binary mobile phase consisting of (A) H₂O+0.1% formic acid and (B) CH₃CN+0.1% formic acid. Method: 0-1 min 5% B, 1-6 min linear gradient to 100% B, 6-7 min 100% B, 7-8 min linear gradient to 5% B, 8-10 min 5% B. Mass spectra were acquired in the continuum mode across the m/z range of 100-1500 at 0.5 s per scan. The operating parameters were as follows: the nebulization gas flow rate was set to 800 L/h with a desolvation temperature of 300° C., the cone gas flow rate was set to 1 L/h, and the ion source temperature was 120° C. The capillary, sampling cone, and extraction cone voltages were tuned to 3 kV, 40 V, and 4 V, respectively. Liquid nitrogen served as a source of nebulizer gas. Data acquisition and instrument control were accomplished using MassLynx software, version 4.1. Samples were infused into the ionization chamber from the UPLC system. All mass spectra were referenced internally using leucine encephalin (2 ng/μL) as the lockspray. The instrument was calibrated periodically using sodium formate following manufacturer's instructions.

Gamma spectra were acquired using a high purity germanium (HPGe) detector controlled using Gamma Vision software (AMETEK Inc.). The detector was calibrated using a multi-source standard containing ²⁴¹Am, ¹⁰⁹Cd, ⁵⁷Co, ¹³⁹Ce, ²⁰³Hg, ¹¹³Sn, ⁸⁵Sr, ¹³⁷Cs, and ⁸⁸Y (Eckert & Zeigler Radiopharma Inc.). Quantification was performed using InterSpec v1.0.9 (National Technology and Engineering Solutions of Sandia, LLC.). For 227Th, the 234.8/236 keV doublet with an absolute γ-ray emission probability (Iγ) of 0.12937 was used and for ²²³Ra, the 154.2 keV peak with Iγ=0.05699 was used. Liquid scintillation counting (LSC) was performed using a Wallac 1414 Guardian liquid scintillation counter controlled with WinSpectral software (Perkin Elmer, Inc.). Samples were prepared by diluting 1 μL of metal stock solution in 0.1 M HCl into 5 mL of Ultima Gold liquid scintillation cocktail (Perkin Elmer, Inc.) and shaking. Samples were counted for 10 min with α/β separation using the Pulse Shape Analysis function as implemented in WinSpectral.

Instant thin layer chromatography silicic acid (iTLC-SA) paper (Agilent Technologies, Inc.) was cut into 1.5×11.4 cm strips and marked 1 cm from the top (solvent front) and 1.5 cm from the bottom (origin) with a pencil before drying at 150° C. for at least three hours before use.

The ²²⁷Th used in this study was obtained from the decay of a legacy stock of ²²⁷Ac at Lawrence Berkeley National Laboratory. The purification procedures are described in detail below. A solution of [⁸⁹Zr]Zr oxalate was obtained as a solution in 1 M oxalic acid from 3D Imaging, LLC and was used as received.

Synthesis and characterization of S1-S9 is described in the Supporting Information for: “Macrocyclic 1,2-Hydroxypyridinone-based Chelators as Potential Ligands for Thorium-227 and Zirconium-89 Radiopharmaceuticals”, Inorganic Chemistry, Vol. 60 (Issue 50): pp. 20721-20732, 2023 (published Aug. 17, 2023), hereby incorporated by reference.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A metal chelator having a chemical structure comprising: 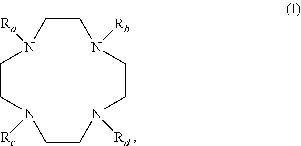
or a bioconjugate thereof; wherein Rₐ, R_{b}, R_{c}, and R_{d} each independently comprises:
(a) a HOPO group having the following chemical structure: 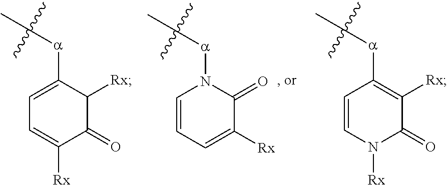
wherein α is a 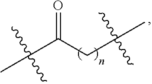
—CHOH—(CH₂)ₙ—, or —(CH₂)ₙ—; each Rₓ is independently a H, —OH or alkyl group; and n is an integer from 0 to 10;
(b) a HA group having the following chemical structure: 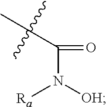
wherein Rα is a —H or alkyl group; or,
(c) a CAM group having the following chemical structure:

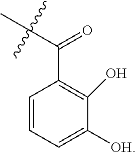 [SMILES: *C(=O)c1cccc(O)c1O]

2. The metal chelator of claim 1, wherein Rₐ, R_{b}, R_{c}, and R_{d} each comprises the HOPO group.

3. The metal chelator of claim 2, wherein the HOPO group is

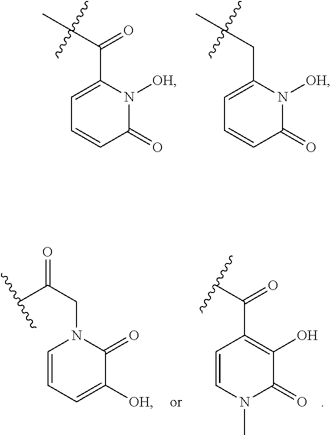 [SMILES: *C(=O)c1cccc(=O)n1O ; *Cc1cccc(=O)n1O ; *C(=O)Cn1cccc(O)c1=O ; *C(=O)c1ccn(C)c(=O)c1O]

4. The metal chelator of claim 3, wherein the metal chelator is

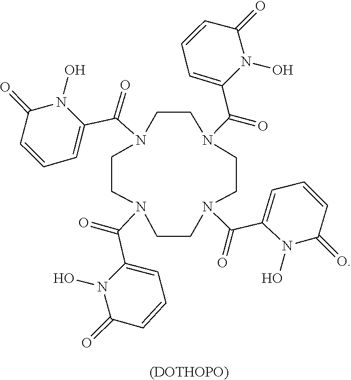

5. A bioconjugated metal chelator comprising metal chelator of the claim 1.

6. The bioconjugated metal chelator of the claim 5, wherein the metal chelator is bound to a biological targeting vector (BTV).

7. The bioconjugated metal chelator of the claim 6, wherein the metal chelator is bound to the BTV via a

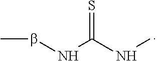

8. The bioconjugated metal chelator of the claim 5, wherein a radioisotope or radionuclide is bound to the metal chelator.

9. The bioconjugated metal chelator of the claim 8, wherein the radioisotope or radionuclide H,—is Thorium-227 and Zirconium-89, or a mixture thereof.

10. A method for treating a subject in need of such treatment, the method comprising:
administering a therapeutically effective amount of a pharmaceutical composition comprising a bioconjugated metal chelator comprising a metal chelator bound to a radioisotope or radionuclide and a biological targeting vector (BTV), wherein the metal chelator has a chemical structure comprising: 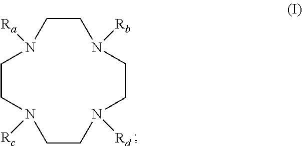
wherein Rₐ, R_{b}, R_{c}, and R_{d} each independently comprises:
(a) a HOPO group having the following chemical structure: 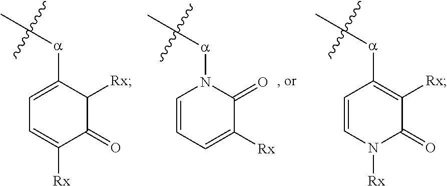
wherein α is a 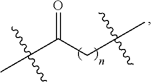
—CHOH—(CH₂)ₙ—, or —(CH₂)ₙ—; each Rₓ is independently a H, —OH or alkyl group; and n is an integer from 0 to 10;
(b) a HA group having the following chemical structure: 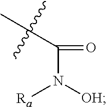
wherein Rα is a —H or alkyl group; or,
(c) a CAM group having the following chemical structure: 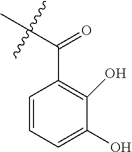 [SMILES: *C(=O)c1cccc(O)c1O]
to the subject; wherein the BTV of the bioconjugated metal chelator targets a cancer cell in the subject.

11. The method of claim 10, wherein the pharmaceutical composition comprises a pharmaceutically acceptable salt.

12. The method of claim 10, wherein Rₐ, R_{b}, R_{c}, and R_{d} each comprises the HOPO group.

13. The method of claim 12, wherein the HOPO group is

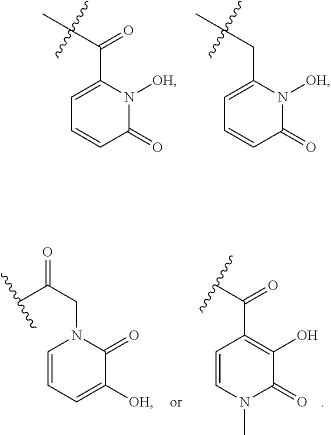 [SMILES: *C(=O)c1cccc(=O)n1O ; *Cc1cccc(=O)n1O ; *C(=O)Cn1cccc(O)c1=O ; *C(=O)c1ccn(C)c(=O)c1O]

14. The method of claim 13, wherein the metal chelator is

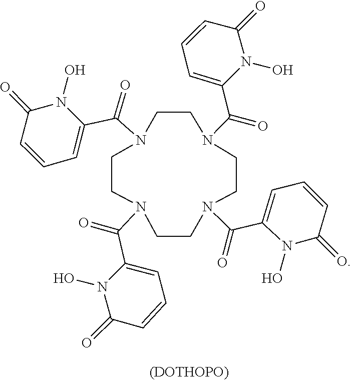

15. The method of claim 10, wherein the metal chelator is bound to the BTV via a

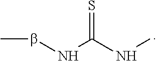

16. The method of claim 10, wherein the radioisotope or radionuclide H,—is Thorium-227 and Zirconium-89, or a mixture thereof.

17. A method for treating a subject for a heavy metal exposure, the method comprising administering a therapeutically effective amount of a pharmaceutical composition comprising the metal chelator of claim 1 to a subject that has an excess amount of one or more of radioisotope or radionuclide, wherein administering results in decorporating, clearing or reducing the amount radioisotope or radionuclide from the subject.

18. A method for the prophylactic treatment of a subject for metal exposure, the method comprising administering a therapeutically effective amount of a pharmaceutical composition comprising the metal chelator of claim 1 to a subject.
